(19) United States
(12) Patent Application Publication
(10) Pub. No.: US 2019/0059362 A1
(43) Pub. Date: Feb. 28, 2019

(54) PERFUSION COMPOSITIONS AND METHODS OF USING ALPHA-1 ANTI-TRYPSIN IN EX VIVO ORGAN PERFUSION

(71) Applicant: CSL Behring LLC, King of Prussia, PA (US)

(72) Inventors: Mingyao Liu, Toronto (CA); Shaf Keshavjee, Toronto (CA); Melvin Berger, King of Prussia, PA (US)

(73) Assignee: CSL Behring LLC, King of Prussia, PA (US)

(21) Appl. No.: 16/113,559

(22) Filed: Aug. 27, 2018

Related U.S. Application Data
(60) Provisional application No. 62/550,333, filed on Aug. 25, 2017.

(51) Int. Cl. A01N 1/02

(52) U.S. Cl. CPC A01N 1/0247; A61K 45/06; A01N 1/0278; A01N 1/0226

(57) ABSTRACT

Perfusion solutions comprising A1AT for the ex vivo perfusion of donor organs are provided to improve donor organ quality and repair damaged donor organs for transplantation. Methods of ex vivo perfusion of donor organs with A1AT-containing perfusion solutions under normothermic temperatures are also provided.

### CROSS-REFERENCE TO RELATED APPLICATION

The present application, filed Monday, Aug. 27, 2018, claims the benefit of priority to U.S. Provisional Application No. 62/550,333, filed Aug. 25, 2017, which is incorporated by reference herein in its entirety for any purpose.

### FIELD

The present disclosure relates to compounds and methods for ex vivo organ perfusion using alpha-1 antitrypsin (A1AT) for improving donor organ quality.

### BACKGROUND

Organ transplantation, including lung transplantation, may be an option for patients with end stage organ diseases. However, many donor organs are discarded prior to transplantation contributing to a high incidence of waitlist deaths. For example, donor lungs may be considered unacceptable for transplant for any number of reasons, including the donor's age, the donor's smoking history, the type of injury leading to death, the presence of pulmonary infection, the length of time between removal from donor and transplantation, etc. See Diamond, J. M., et al., Clinical risk factors for primary graft dysfunction after lung transplantation. Am J Respir Crit Care Med, 2013. 187(5): p. 527-34. Ex vivo lung perfusion (EVLP) has been attempted to repair injured donor lungs and expand the pool of acceptable donor lungs. See Machuca and Cypel, Ex vivo lung perfusion, J Thorac Dis, 2014 6(8): p. 1054-62.

Donor organs may also be injured during the transplantation process. For instance, a major cause of death during the early post-operative period following lung transplantation is primary graft dysfunction (PGD). Ischemia-reperfusion (IR) injury of the lung can occur when the blood supply returns to the lung after a period of ischemia, and is thought to contribute to PGD. Inflammation and cell death have been identified as underlying mechanisms of IR injury. While some protection from ischemia may be achieved with cold flush preservation and cellular metabolism may be slowed by cold storage of the donor organ, prolonged hypothermic preservation may be deleterious to the lung. And with a continual shortage of acceptable donor lungs, there is a need for new compositions and methods for maintaining and improving donor lung quality and the quality of other transplantable organs.

Alpha-1 antitrypsin (A1AT) is a protease inhibitor and member of the serpin family of proteins. It is currently indicated for A1AT deficiency replacement therapy, for example. A1AT binds to enzymatic targets such as neutrophil elastase and has been shown to have anti-inflammatory, anti-neutrophil influx and activation, and anti-apoptotic effects on cells. Ex vivo perfusion of donor organs, including donor lungs, using A1AT-containing perfusion solutions under normothermic temperatures may improve donor organ quality and repair damaged donor organs for transplantation.

### SUMMARY

The present disclosure includes, for example, methods of ex vivo organ perfusion, comprising perfusing an organ ex vivo with a perfusion solution comprising alpha-1-antitrypsin (A1AT), wherein the perfusion solution is exposed to an environment having a normothermic temperature. The present disclosure also includes, for example, methods of transplanting an organ, comprising: perfusing an organ ex vivo with a perfusion solution comprising A1AT, wherein the perfusion solution is exposed to an environment having a normothermic temperature; and transplanting the ex vivo perfused organ into a recipient.

In some embodiments of the transplantation methods, A1AT is further administered to the organ recipient before the organ is transplanted or during organ transplantation. In some embodiments of the transplantation methods, A1AT is further administered to the organ recipient after the organ is transplanted. In some embodiments of the transplantation methods, at least one additional therapeutic agent other than A1AT is administered to the organ recipient before, during, or after the organ is transplanted, wherein the agent is one or more of interferon, an interferon derivative, a prostane derivative, a glucocorticoid, an immunosuppressive, a lipoxygenase inhibitor, a leukotriene antagonist, a soluble TNF-receptor, an anti-TNF antibody, a soluble receptor of an interleukin, a cytokine, a T-cell protein, an anti-interleukin receptor antibody, an anti-CD20 antibody, a C1 esterase inhibitor, an interleukin antagonist (such as an anti-interleukin antibody), an IL6 antagonist (such as an anti-IL6R antibody), IL6R antagonist (such as an anti-IL6R antibody), IDeS, an IVIG, an SCIG, an anti-CD25/IL-2 receptor antibody, and/or an anti-thymocyte globulin (ATG). In some embodiments, the additional therapeutic agent is one or more of betaseron, beta-interferon, iloprost, cicaprost, cortisol, prednisolone, methyl-prednisolone, dexamethasone, cyclosporine A, FK-506, methoxsalene, thalidomide, sulfasalazine, azathioprine, methotrexate, zileutone, MK-886, WY-50295, SC-45662, SC-41661A, BI-L-357, adrenocorticotropic hormone (ACTH), an ACTC analog, calcipotriol, mycophenolate mofetil, a mycophenolate mofetil analog, rituximab, basiliximab, daclizumab, alemtuzumab, and/or ATG. In some embodiments, the additional therapeutic agent is one or more of C1 esterase inhibitor, IVIG, SCIG, basiliximab, daclizumab, alemtuzumab, and/or ATG. In some embodiments, the A1AT and/or additional therapeutic agent is administered to the organ recipient intravenously or subcutaneously.

In any of the methods above, the organ may be a lung, a heart, a liver, a kidney, a pancreas, or a small bowel. In some embodiments, the organ is a lung. In some embodiments, the lung is ventilated during ex vivo lung perfusion.

Methods of this disclosure also include methods of ex vivo lung perfusion, comprising perfusing a lung ex vivo with a perfusion solution comprising A1AT and ventilating the lung. In some embodiments, the perfusion solution is exposed to an environment having a normothermic temperature.

In any of the above methods, the perfusion solution may be exposed to an environment having a normothermic temperature of from 30° C. to 38° C., of from 32° C. to 38° C., of from 34° C. to 38° C., of from 36° C. to 38° C., of 30° C., of 31° C., of 32° C., of 33° C., of 34° C., of 35° C., of 36° C., of 37° C., or of 38° C. In any of the above methods, the environment may be a vessel, a circuit, a heat-transfer fluid, or a reservoir comprising a heat-transfer fluid. In any of the above methods wherein the organ is a lung, the lung may be ventilated with gas having an oxygen content of from 20% to 100%, of from 25% to 90%, of from 30% to 80%, of from 40% to 70%, of from 50% to 60%, of 21%, or of 100%. In any of the above methods, the organ may be perfused for a period of time of from 1 hour to 6 hours, of from 1 hour to 12 hours, of from 1 hour to 13 hours, of from 1 hour to 14 hours, of from 1 hour to 15 hours, of from 1 hour to 16 hours, of from 1 hour to 18 hours, of from 1 hour to 20 hours, of from 1 hour to 24 hours, of from 1 hour to 36 hours, of from 1 hour to 48 hours, of from 1 hour to 60 hours, of from 1 hour to 72 hours, of at least 4 hours, of at least 6 hours, of at least 7 hours, of at least 8 hours, of at least 8 hours, of at least 10 hours, of at least 11 hours, of at least 12 hours, of from 6 hours to 12 hours, of from 6 hours to 13 hours, of from 6 hours to 14 hours, of from 6 hours to 15 hours, of from 6 hours to 16 hours, of from 6 hours to 18 hours, of from 6 hours to 20 hours, of from 6 hours to 24 hours, of from 6 hours to 36 hours, of from 6 hours to 48 hours, of from 6 hours to 60 hours, of from 6 hours to 72 hours, of from 12 hours to 24 hours, of from 12 hours to 36 hours, of from 12 hours to 48 hours, of from 12 hours to 60 hours, of from 12 hours to 72 hours, of from 24 hours to 36 hours, of from 24 hours to 42 hours, of from 24 hours to 60 hours, of from 24 hours to 72 hours, of from 36 to 42 hours, of from 36 to 48 hours, of from 36 to 60 hours, of 1 hour, of 2 hours, of 3 hours, of 4 hours, of 5 hours, of 6 hours, of 10 hours, of 12 hours, of 15 hours, of 18 hours, of 20 hours, of 24 hours, of 30 hours, of 36 hours, of 42 hours, of 48 hours, of 54 hours, of 60 hours, of 66 hours, of 72 hours, at least 1 day, at least 2 days, at least 3 days, at least 4 days, at least 5 days, at least 6 days, at least 7 days.

In any of the above methods, the organ may have been donated after brain death (DBD) or after cardiac death (DCD).

In any of the above methods, before perfusing, the organ may be processed, preserved, or stored in a below room temperature environment. In such methods, after perfusing and prior to transplantation, the organ may be processed, preserved, or stored in a below room temperature environment. In such methods, organ may be processed, preserved, or stored in an environment having a temperature of from 4° C. to 12° C., of from 4° C. to 8° C., of 4° C. to 10° C., of 4° C., of 5° C., of 6° C., of 7° C., of 8° C., of 9° C., of 10° C., of 11° C., or of 12° C. before and/or after perfusion. For example, the organ may be processed, preserved, or stored in an environment having a temperature of 4° C. before and/or after perfusion. For example, the organ may be processed, preserved, or stored in a below room temperature environment before and/or after perfusion for a period of time of from 1 hour to 6 hours, of from 1 hour to 12 hours, of from 1 hour to 24 hours, of from 1 hour to 36 hours, of from 1 hour to 48 hours, of from 1 hour to 60 hours, of from 1 hour to 72 hours, of from 6 hours to 12 hours, of from 6 hours to 24 hours, of from 6 hours to 36 hours, of from 6 hours to 48 hours, of from 6 hours to 6 hours, of from 6 hours to 72 hours, of from 12 hours to 24 hours, of from 12 hours to 36 hours, of from 12 hours to 48 hours, of from 12 hours to 60 hours, of from 12 hours to 72 hours, of from 24 hours to 36 hours, of from 24 hours to 42 hours, of from 24 hours to 60 hours, of from 24 hours to 72 hours, of from 36 to 42 hours, of from 36 to 48 hours, of from 36 to 60 hours, of 1 hour, of 2 hours, of 3 hours, of 4 hours, of 5 hours, of 6 hours, of 10 hours, of 12 hours, of 15 hours, of 18 hours, of 20 hours, of 24 hours, of 30 hours, of 36 hours, of 42 hours, of 48 hours, of 54 hours, of 60 hours, of 66 hours, or of 72 hours. In any of these methods, the processing, preservation, and/or storage may comprise exposing or perfusing the organ with a solution that does not comprise A1AT.

In any of the methods of this disclosure, the A1AT may comprise human A1AT. In any of the methods, the A1AT may be obtained from pooled human plasma, is recombinant, or is a fusion molecule comprising A1AT and a fusion partner, wherein the fusion partner optionally comprises an Fc variant. In any of the methods disclosed herein, the concentration of A1AT in the perfusion solution may be from 0.5 mg/mL to 5 mg/mL, from 0.5 mg/mL to 10 mg/mL, from 1 mg/mL to 5 mg/mL, from 3 mg/mL to 10 mg/mL, from 5 mg/mL to 10 mg/mL, from 5 mg/mL to 15 mg/mL, from 8 mg/mL to 12 mg/mL, from 10 mg/mL to 20 mg/mL, from 10 mg/mL to 15 mg/mL, or from 15 mg/mL to 20 mg/mL. In any of the methods herein, the concentration of A1AT in the perfusion solution may be 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL.

In any of the methods disclosed herein, the perfusion solution may comprise albumin, such as human serum albumin. If albumin is included, the concentration of albumin may be from 50 mg/mL to 100 mg/mL, from 55 mg/mL to 85 mg/mL, from 60 mg/mL to 80 mg/mL, from 65 mg/mL to 85 mg/mL, from 70 mg/mL to 80 mg/mL, 50 mg/mL, 60 mg/mL, 65 mg/mL, 70 mg/mL, 75 mg/mL, 80 mg/mL, of 85 mg/mL, 90 mg/mL, or 100 mg/mL. In any of the methods herein, the perfusion solution may comprise a polysaccharide compound or a sugar compound, for example, a dextran compound. In such cases, a dextran compound may comprise a dextran having a molecular weight of from 1 kDa to 250 kDa, of from 20 kDa to 150 kDa, of from 50 kDa to 100 kDa, of 1 kDa, of 5 kDa, of 10 kDa, of 20 kDa, of 30 kDa, of 40 kDa, of 50 kDa, of 60 kDa, of 70 kDa, of 80 kDa, of 90 kDa, of 100 kDa, of 150 kDa, of 200 kDa, or of 250 kDa. For instance, the dextran compound may comprise a dextran having a molecular weight of 40 kDa. The concentration of the dextran compound may be from 1 mg/mL to 55 mg/mL, from 2 mg/mL to 25 mg/mL, from 2 mg/mL to 20 mg/mL, from 2 mg/mL to 10 mg/mL, from 2 mg/mL, 5 mg/mL, 10 mg/mL, 20 mg/mL, or 25 mg/mL. For instance, the concentration of dextran compound may be 5 mg/mL.

In methods disclosed herein, the perfusion solution may comprise a one or a combination of salts. In some embodiments, the salt(s) comprise sodium ion, potassium ion, calcium ion, magnesium ion, hydrogen carbonate ion, chloride ion, sodium chloride, potassium chloride, calcium chloride, magnesium chloride, sodium dihydrogen phosphate, sodium bicarbonate, or sodium hydroxide. For example, the concentration of each salt may correspond to its normal serum concentration in human or animal blood. In some embodiments, a combination of salts may comprise sodium ion at a concentration of from 135 mM to 150 mM, potassium ion at a concentration of from 3 mM to 5 mM, chloride ion at a concentration of from 95 mM to 110 mM, hydrogen carbonate ion at a concentration of from 20 mM to 30 mM, calcium ion at a concentration of from 2 to 3 mM, or phosphate ion at a concentration of from 1 mM to 1.5 mM. In some embodiments, a combination of salts may comprise sodium chloride at a concentration of from 75 mM to 150 mM, potassium chloride at a concentration of from 0.4 mM to 5 mM, calcium chloride at a concentration of from 1 mM to 2 mM, magnesium sulfate at a concentration of from 1 mM to 2 mM, or sodium bicarbonate at a concentration of from 10 mM to 20 mM.

In any of the methods disclose herein, at least one physiological function or at least one biological marker of the donor organ is measured before the ex vivo perfusion, during the ex vivo perfusion, at the termination of the ex vivo perfusion, or at a time after the ex vivo perfusion. In some such embodiments, the physiological function at the termination of or at a time after the ex vivo perfusion is increased or improved compared to the physiological function at a time before or during the ex vivo perfusion. In some embodiments, the physiological function at the termination of or at a time after the ex vivo perfusion is decreased compared to the physiological function at a time before or during the ex vivo perfusion. In some embodiments, the organ is a lung and the pulmonary arterial pressure (PAP) at the termination of the ex vivo perfusion or at a time after the ex vivo perfusion is decreased compared to the PAP at a time before or during the ex vivo perfusion. In some embodiments, the organ is a lung and the pulmonary vascular resistance (PVR) at the termination of the ex vivo perfusion or at a time after the ex vivo perfusion is decreased compared to the PVR at a time before or during the ex vivo perfusion. In some embodiments, the organ is a lung and the peak inspiratory pressure (Ppeak) sampled at the termination of the ex vivo perfusion or at a time after the ex vivo perfusion is decreased compared to the Ppeak sampled at a time before or during the ex vivo perfusion. In some embodiments, the organ is a lung and the plateau airway pressure (Pplat) sampled at the termination of the ex vivo perfusion or at a time after the ex vivo perfusion is decreased compared to the Pplat sampled at a time before or during the ex vivo perfusion. In some embodiments, the organ is a lung and the dynamic pulmonary compliance (Cdyn) sampled at the termination of the ex vivo perfusion or at a time after the ex vivo perfusion is (a) increased compared to the Cdyn sampled at a time before or during the ex vivo perfusion, or (b) maintained compared to the Cdyn sampled at a time before or during the ex vivo perfusion. In some embodiments, the organ is a lung and the static pulmonary compliance (Cstat) sampled at the termination of the ex vivo perfusion or at a time after the ex vivo perfusion is (a) increased compared to the Cstat sampled at a time before or during the ex vivo perfusion, or (b) maintained compared to the Cstat sampled at a time before or during the ex vivo perfusion. In some embodiments, the organ is a lung and the ratio of arterial oxygen partial pressure to fractional inspired oxygen (PaO₂/FiO₂) at the termination of the ex vivo perfusion or at a time after the ex vivo perfusion is (a) increased compared to the PaO₂/FiO₂ at a time before or during the ex vivo perfusion, or (b) maintained compared to the PaO₂/FiO₂ at a time before or during the ex vivo perfusion. In some embodiments, the organ is a lung and the ratio of arterial oxygen partial pressure to fractional inspired oxygen (PaO₂/FiO₂) before the ex vivo perfusion is less than 250 mmHg, less than 275 mmHg, less than 300 mmHg, less than 325 mmHg, less than 350 mmHg, or less than 400 mmHg. In some embodiments, the organ is a lung and the ratio of arterial oxygen partial pressure to fractional inspired oxygen (PaO₂/FiO₂) during the ex vivo perfusion, at the termination of the ex vivo perfusion, or at a time after the ex vivo perfusion is at least 300 mmHg or greater, at least 325 mmHg or greater, at least 350 mmHg or greater, at least 375 mmHg or greater, at least 400 mmHg or greater, at least 425 mmHg or greater, at least 450 mmHg or greater, at least 475 mmHg or greater, or at least 500 mmHg or greater. In some embodiments, PaO₂/FiO₂ is measured by the oxygen pressure (PO₂) in the left atrium. In some embodiments, the organ is a lung and the difference in oxygen pressure (PO₂) between the left atrium (LA) and pulmonary artery (PA) (Delta PO₂) at the termination of the ex vivo perfusion or at a time after the ex vivo perfusion is (a) increased compared to the Delta PO₂ at a time before or during the ex vivo perfusion, or (b) maintained compared to the Delta PO₂ at a time before or during the ex vivo perfusion. In some embodiments, the organ is a lung and the levels of caspase 3, IL-8, TNF-alpha, TREM-1, IL-6, IL-1b, GM-CSF, endothelin, and/or IL-10 are measured in lung tissue at the end of ex vivo perfusion and compared to levels before the start of ex vivo perfusion.

The present disclosure also provides a perfusion solution comprising (a) albumin, (b) a dextran compound, (c) a combination of salts, and (d) alpha-1-antitrypsin (A1AT). In some embodiments, the A1AT comprises human A1AT. In some embodiments, the A1AT is obtained from pooled human plasma, is recombinant, or is a fusion molecule comprising A1AT and a fusion partner, wherein the fusion partner optionally comprises an Fc variant. In some embodiments, the concentration of A1AT is from 0.5 mg/mL to 5 mg/mL, from 0.5 mg/mL to 10 mg/mL, from 1 mg/mL to 5 mg/mL, from 3 mg/mL to 10 mg/mL, from 5 mg/mL to 10 mg/mL, from 5 mg/mL to 15 mg/mL, from 8 mg/mL to 12 mg/mL, from 10 mg/mL to 20 mg/mL, from 10 mg/mL to 15 mg/mL, or from 15 mg/mL to 20 mg/mL. In some embodiments, the concentration of A1AT is 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL. In some embodiments, the albumin comprises human serum albumin. In some embodiments, the concentration of albumin is from 50 mg/mL to 100 mg/mL, from 55 mg/mL to 85 mg/mL, from 60 mg/mL to 80 mg/mL, from 65 mg/mL to 85 mg/mL, from 70 mg/mL to 80 mg/mL, 50 mg/mL, 60 mg/mL, 65 mg/mL, 70 mg/mL, 75 mg/mL, 80 mg/mL, of 85 mg/mL, 90 mg/mL, or 100 mg/mL. In some embodiments, the dextran compound comprises a dextran having a molecular weight of from 1 kDa to 250 kDa, of from 20 kDa to 150 kDa, of from 50 kDa to 100 kDa, of 1 kDa, of 5 kDa, of 10 kDa, of 20 kDa, of 30 kDa, of 40 kDa, of 50 kDa, of 60 kDa, of 70 kDa, of 80 kDa, of 90 kDa, of 100 kDa, of 150 kDa, of 200 kDa, or of 250 kDa. In some embodiments, the dextran compound comprises a dextran having a molecular weight of 40 kDa. In some embodiments, the concentration of dextran compound is from 1 mg/mL to 55 mg/mL, from 2 mg/mL to 25 mg/mL, from 2 mg/mL to 20 mg/mL, from 2 mg/mL to 10 mg/mL, 2 mg/mL, 5 mg/mL, 10 mg/mL, 20 mg/mL, or 25 mg/mL. In some embodiments, the concentration of dextran compound is 5 mg/mL. In some embodiments, the combination of salts comprises sodium ion, potassium ion, calcium ion, magnesium ion, hydrogen carbonate ion, chloride ion, sodium chloride, potassium chloride, calcium chloride, magnesium chloride, sodium dihydrogen phosphate, sodium bicarbonate, or sodium hydroxide. In some embodiments, the concentration of each salt corresponds to its normal serum concentration in human or animal blood. In some embodiments, the combination of salts comprises sodium ion at a concentration of from 135 mM to 150 mM, potassium ion at a concentration of from 3 mM to 5 mM, chloride ion at a concentration of from 95 mM to 110 mM, hydrogen carbonate ion at a concentration of from 20 mM to 30 mM, calcium ion at a concentration of from 2 to 3 mM, or phosphate ion at a concentration of from 1 mM to 1.5 mM. In some embodiments, the combination of salts comprises sodium chloride at a concentration of from 75 mM to 150 mM, potassium chloride at a concentration of from 0.4 mM to 5 mM, calcium chloride at a concentration of from 1 mM to 2 mM, magnesium sulfate at a concentration of from 1 mM to 2 mM, or sodium bicarbonate at a concentration of from 10 mM to 20 mM. In some embodiments, the concentration of each salt corresponds to its normal serum concentration in human or animal blood.

### BRIEF DESCRIPTION OF THE DRAWINGS

FIGS. 1A and 1B show the mean pulmonary arterial pressure (PAP) (FIG. 1A) and the mean pulmonary vascular resistance (PVR) (FIG. 1B) of pig donor lungs during EVLP from an A1AT-treatment group and a control group.

FIGS. 2A and 2B show the mean peak inspiratory pressure (Ppeak, FIG. 2A) and plateau airway pressure (Pplat, FIG. 2B) of pig donor lungs during the EVLP for the A1AT-treatment group and the control group.

FIGS. 3A and 3B show the mean change in baseline Ppeak (FIG. 3A) and in baseline Pplat (FIG. 3B) of pig donor lungs measured at 1 hour of EVLP (baseline) and at the end of EVLP (12 hours) for the A1AT-treatment group and the control group.

FIGS. 4A and 4B show the mean dynamic pulmonary compliance (Cdyn, FIG. 4A) and static pulmonary compliance (Cstat, FIG. 4B) of pig donor lungs during the EVLP for the A1AT-treatment group and the control group.

FIGS. 5A and 5B show the mean change in baseline Cdyn (FIG. 5A) and in baseline Cstat (FIG. 5B) of pig donor lungs measured at 1 hour of EVLP (baseline) and at the end of EVLP (12 hours) for the A1AT-treatment group and the control group.

FIGS. 6A and 6B show the mean ratio of arterial oxygen partial pressure to fractional inspired oxygen (PO₂/FiO₂, FIG. 6A) and difference in PO₂ between left atrium and pulmonary artery (Delta PO₂) of pig donor lungs during the EVLP for the A1AT-treatment group and the control group.

FIGS. 7A and 7B show the mean change in the baseline left atrium PO₂/FiO₂ (FIG. 7A) and in the baseline delta PO₂ (FIG. 7B) of pig donor lungs measured at 1 hour of EVLP (baseline) and at the end of EVLP (12 hours) for the A1AT-treatment group and the control group.

FIG. 8 shows the mean wet-to-dry lung weight ratio of pig donor lungs at the end of EVLP in the A1AT treatment group and the control group.

FIG. 9A is a representative TUNEL staining image at 200× magnification of a control lung at the end of EVLP.

FIG. 9B is a representative TUNEL staining image at 200× magnification of an A1AT-treated lung at the end of EVLP.

FIG. 9C shows the mean ratio of TUNEL-positive cells to total cells in the control group and the A1AT-treatment group.

FIGS. 10A-F show the mean concentration of IL-4 (FIG. 10A), IL-6 (FIG. 10B), IL-12 (FIG. 10C), IL-18 (FIG. 10D), IL-10 (FIG. 10E), IL-1 ra (FIG. 10F) in the EVLP perfusate at 1 hour, 3 hours, 5 hours, 7 hours, 9 hours, 11 hours, and 12 hours of EVLP for the control group and the A1AT-treatment group.

FIGS. 11A-E show the mean concentration of IL-1β (FIG. 11A), IL-2 (FIG. 11B), TNF-α (FIG. 11C), IL-1α (FIG. 11D), and IL-8 (FIG. 11E) in the EVLP perfusate at 1 hour, 3 hours, 5 hours, 7 hours, 9 hours, 11 hours, and 12 hours of EVLP for the control group and the A1AT-treatment group.

FIGS. 12A-D show the mean concentration of sodium ion (FIG. 12A), potassium ion (FIG. 12B), chloride ion (FIG. 12C), and calcium ion (FIG. 12D) in the EVLP perfusate at 1 hour, 2 hours, 3 hours, 4 hours, 5 hours, 6 hours, 7 hours, 8 hours, 9 hours, 10 hours, 11 hours, and 12 hours for the control group and the A1AT-treatment group.

FIG. 13 shows the delta pO₂ during EVLP for human ex vivo lung perfusion case 1 described in Example 13 below.

FIGS. 14A-B show the dynamic compliance (FIG. 14A) and static compliance (FIG. 14B) during EVLP for case 1.

FIGS. 15A-C show A1AT levels in perfusate and tissue for case 1, specifically, A1AT levels in left and right perfusate (FIG. 15A), A1AT levels in tissue (FIG. 15B), and A1AT concentration in left and right perfusate (FIG. 15C).

FIGS. 16A-B show caspase 3 levels over time (FIG. 16A) and at the end of perfusion after 12 hours in the treatment and control lungs (FIG. 16B) for case 1.

FIGS. 17A-D show levels of cytokines in perfusate (in pg/mL) for treatment and control lungs of case 1, specifically: TNF alpha (FIG. 17A), IL-8 (FIG. 17B), TREM-1 (FIG. 17C), and IL-6 (FIG. 17D).

FIG. 18 shows the delta pO₂ during EVLP for human ex vivo lung perfusion case 2 described in Example 13 below.

FIGS. 19A-C show the peak airway pressure (FIG. 19A), dynamic compliance (FIG. 19B) and static compliance (FIG. 19C) during EVLP for case 2.

FIGS. 20A-B show A1AT levels in perfusate (FIG. 20A) and tissue (FIG. 20B) for case 2, respectively.

FIGS. 21A-B show caspase 3 levels over time (FIG. 21A) and at the end of perfusion after 12 hours in the treatment and control lungs (FIG. 21B) for case 2.

FIG. 22 shows neutrophil elastase (NE) content in bronchial wash of the donor lungs before EVLP and of each lung (treatment and control) after EVLP.

FIGS. 23A-D show levels of cytokines in perfusate (in pg/mL) for treatment and control lungs of case 2 (not corrected for dilution), specifically: IL-6 (FIG. 23A), CXCL8 (FIG. 23B), TNF alpha (FIG. 23C), and TREM-1 (FIG. 23D).

FIGS. 24A-B show the delta pO₂ during EVLP (FIG. 24A) and Steen® loss (FIG. 24B) for human case 3 described in Example 13 below.

FIGS. 25A-C show the peak airway pressure (FIG. 25A), static compliance (FIG. 25B) and dynamic compliance (FIG. 25C) during EVLP for case 3.

FIGS. 26A-H show levels of cytokines in perfusate (in pg/mL) for treatment and control lungs of case 3 (not corrected for dilution), specifically: IL-6 (FIG. 26A), IL-8 (FIG. 26B), IL-1b (FIG. 26C), TNF alpha (FIG. 26D), TREM-1 (FIG. 26E), GM-CSF (FIG. 26F), endothelin (FIG. 26G), and IL-10 (FIG. 26H).

FIGS. 27A-B show the delta pO₂ during EVLP (FIG. 27A) and Steen® loss (FIG. 27B) for human case 4 described in Example 13 below.

FIGS. 28A-C show the peak airway pressure (FIG. 28A), static compliance (FIG. 28B) and dynamic compliance (FIG. 28C) during EVLP for case 4.

FIGS. 29A-H show levels of cytokines in perfusate (in pg/mL) for treatment and control lungs of case 4 (not corrected for dilution), specifically: IL-6 (FIG. 29A), IL-8 (FIG. 29B), IL-1b (FIG. 29C), TNF alpha (FIG. 29D), TREM-1 (FIG. 29E), GM-CSF (FIG. 29F), endothelin (FIG. 29G), and IL-10 (FIG. 29H).

FIGS. 30A-B show the delta pO₂ during EVLP (FIG. 30A) and Steen® loss (FIG. 30B) for human case 5 described in Example 13 below.

FIGS. 31A-C show the peak airway pressure (FIG. 31A), static compliance (FIG. 31B) and dynamic compliance (FIG. 31C) during EVLP for case 5.

FIGS. 32A-G show levels of cytokines in perfusate (in pg/mL) for treatment and control lungs of case 5 (not corrected for dilution), specifically: IL-6 (FIG. 32A), IL-8 (FIG. 32B), IL-1b (FIG. 32C), TNF alpha (FIG. 32D), TREM-1 (FIG. 32E), endothelin (FIG. 32F), and IL-10 (FIG. 32G).

### DESCRIPTION OF CERTAIN EMBODIMENTS

Perfusion solutions comprising A1AT for the ex vivo perfusion of donor organs, for example, donor lungs, heart, liver, kidney, pancreas, and small bowel, are described, which, in some embodiments, may improve donor organ quality and repair damaged donor organs for transplantation. Methods of ex vivo perfusion of donor organs with A1AT-containing perfusion solutions under normothermic temperatures are also provided.

As used herein, numerical terms are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

References cited herein are incorporated by reference in their entirety.

### EVLP Solutions Comprising A1AT

Novel donor organ perfusion solutions are provided, for example, perfusion solutions containing A1AT for use in an ex vivo organ perfusion method under normothermic temperatures.

A “perfusion solution” refers to a liquid mixture that is passed through an organ or tissue. The term “perfusate” is also used herein and refers to a perfusion solution after it has been passed through an organ or tissue.

In some embodiments, a perfusion solution comprises A1AT for use in an ex vivo organ perfusion method under normothermic temperatures. In some embodiments, a perfusion solution comprises A1AT, albumin, a dextran compound, and a combination of salts. In some embodiments, a perfusion solution comprises A1AT and STEEN™ Solution. In some embodiments, a perfusion solution comprises A1AT and a solution described in U.S. Pat. No. 7,255,983.

“Alpha-1 antitrypsin” or “A1AT” refers to a polypeptide comprising full length A1AT, an A1AT fusion molecule comprising A1AT and a fusion partner, such as an Fc polypeptide, or a fragment of A1AT that retains at least one protease inhibiting function.

A1AT may be from any vertebrate source, including mammals such as primates (e.g., humans and cynomolgus monkeys), rodents (e.g., mice and rats), and livestock (e.g., bovine and porcine), unless otherwise indicated. In some embodiments, the A1AT may be plasma-derived. In some embodiments, the A1AT may be recombinant. In some embodiments, the A1AT is human A1AT, including recombinant human A1AT or MAT obtained from pooled human plasma. In some embodiments, the A1AT comprises a signal polypeptide whereas in other embodiments it does not. In some embodiments, the A1AT comprises Zemaira® (CSL Behring), Prolastin® (Grifols), Prolastin® C (Grifols), Aralast® (Shire), Aralast NP® (Shire), Glassia® (Kamada), Trypsone® (Grifols), Alfalastin (LFB Biomedicaments), or other commercial formulation or any combination thereof. In some embodiments, the A1AT is a fusion molecule comprising an A1AT and a fusion partner, optionally an Fc molecule, (e.g., an Fc fragment, an Fc analog, etc.), PEG, or albumin, such as an A1AT-Fc fusion molecule described in WO2013/106589 and WO2014/160768.

In some embodiments, the perfusion solution comprises albumin. “Albumin” refers to a polypeptide comprising full-length albumin or a functional fragment of albumin and may be from any vertebrate source, including mammals such as primates (e.g., humans and cynomolgus monkeys), rodents (e.g., mice and rats), and livestock (e.g., bovine and porcine), unless otherwise indicated. The albumin may be purified from a human or animal serum source or the albumin may be made by genetic engineering. In some embodiments, the albumin is human serum albumin, bovine serum albumin, or porcine serum albumin.

In some embodiments, a perfusion solution comprises albumin, such as human serum albumin, at a concentration of from 50 mg/mL to 100 mg/mL, of from 55 mg/mL to 85 mg/mL, of from 60 mg/mL to 80 mg/mL, of from 65 mg/mL to 85 mg/mL, of from 70 mg/mL to 80 mg/mL, of 50 mg/mL, of 60 mg/mL, of 65 mg/mL, of 70 mg/mL, of 75 mg/mL, of 80 mg/mL, of 85 mg/mL, of 90 mg/mL, or of 100 mg/mL.

In some embodiments, the perfusion solution comprises a polysaccharide compound or a sugar compound such as a dextran compound. A “dextran compound” refers to a synthetic dextran molecule composed of glucose units in a chain and comprising glucose side chains or derivatives thereof. The longer the dextran molecule, the higher its molecular weight.

In some embodiments, the dextran compound has a molecular weight of from 1 kDa to 250 kDa, of from 20 kDa to 150 kDa, of from 50 kDa to 100 kDa, of 1 kDa, of 5 kDa, of 10 kDa, of 20 kDa, of 30 kDa, of 40 kDa, of 50 kDa, of 60 kDa, of 70 kDa, of 80 kDa, of 90 kDa, of 100 kDa, of 150 kDa, of 200 kDa, or of 250 kDa. In some embodiments, the dextran compound is Dextran 40, which has a molecular weight of 40 kDa. In some embodiments, the dextran compound is Dextran 50, Dextran 60, or Dextran 70, which have a molecular weight of 50 kDa, 60 kDa, and 70 kDa, respectively.

In some embodiments, a perfusion solution comprises a dextran compound, such as Dextran 40, at a concentration of from 1 mg/mL to 55 mg/mL, of from 2 mg/mL to 25 mg/mL, of from 2 mg/mL to 20 mg/mL, of from 2 mg/mL to 10 mg/mL, of 2 mg/mL, of 5 mg/mL, of 10 mg/mL, of 20 mg/mL, or of 25 mg/mL. In some embodiments, when a dextran compound having a lower molecular weight is used (e.g., Dextran 1, etc.), the concentration used may be greater (e.g., of 10 mg/mL to 150 mg/mL).

In some embodiments, the perfusion solution comprises a salt. A “salt,” as used herein, refers to an ionic compound or ions (cations and/or anions) that may form an ionic compound. For example, a salt includes sodium chloride, sodium ions, or chloride ions.

In some embodiments, a perfusion solution comprises a combination of salts selected from sodium ion, potassium ion, calcium ion, magnesium ion, hydrogen carbonate ion, or chloride ion. In some embodiments, a perfusion solution comprises a combination of salts selected from sodium chloride, potassium chloride, calcium chloride, magnesium chloride, sodium dihydrogen phosphate, sodium bicarbonate, or sodium hydroxide.

In some embodiments, a perfusion solution comprises a combination of salts, wherein the concentration of each salt corresponds to its normal serum concentration in human or animal blood. In some embodiments, a perfusion solution comprises sodium ion at a concentration of from 135 mM to 150 mM, potassium ion at a concentration of from 3 mM to 5 mM, chloride ion at a concentration of from 95 mM to 110 mM, hydrogen carbonate ion at a concentration of from 20 mM to 30 mM, calcium ion at a concentration of from 2 mM to 3 mM, and phosphate ion at a concentration of from 1 mM to 1.5 mM. See Ch. 26-3, Electrolyte Balance, in Anatomy & Physiology, copyright 2013 by Rice University, pp. 1199-1204, available at the following Internet address: solr (dot) bccampus (dot) ca:8001/bcc/file/f4873e49-e09c-469e-9ee8-9f14ca5a4e00/1/AnatomyPhysiology-OpenStax (dot) pdf, which is incorporated herein by reference in its entirety.

In some embodiments, a perfusion solution comprises sodium chloride at a concentration of from 75 mM to 150 mM, potassium chloride at a concentration of from 0.4 mM to 5 mM, calcium chloride at a concentration of from 1 mM to 2 mM, magnesium sulfate at a concentration of from 1 mM to 2 mM, or sodium bicarbonate at a concentration of from 10 mM to 20 mM. See U.S. Pat. No. 7,255,983.

In some embodiments, ex vivo organ perfusion takes place at a normothermic temperature. A “normothermic temperature,” as used herein, refers to an average temperature at or around normal body temperature, including from 30° C. to 38° C., such as 30° C., 31° C., 32° C., 33° C., 34° C., 35° C., 36° C., 37° C., or 38° C. In some embodiments, a perfusion solution has a normothermic temperature of from 30° C. to 38° C., of from 32° C. to 38° C., of from 34° C. to 38° C., of from 36° C. to 38° C., of 30° C., of 31° C., of 32° C., of 33° C., of 34° C., of 35° C., of 36° C., of 37° C., or of 38° C. In some embodiments, a perfusion solution is exposed to an environment having a normothermic temperature of from 30° C. to 38° C., of from 32° C. to 38° C., of from 34° C. to 38° C., of from 36° C. to 38° C., of 30° C., of 31° C., of 32° C., of 33° C., of 34° C., of 35° C., of 36° C., of 37° C., or of 38° C. In some embodiments, an environment having a normothermic temperature may be a vessel (i.e. a physical container), a circuit, a heat-transfer fluid (e.g., water or other aqueous solution), or a reservoir (e.g., in a vessel) comprising a heat-transfer fluid.

### Ex Vivo Perfusion Methods Using A1AT-Containing Perfusion Solutions

Methods of ex vivo organ perfusion comprising perfusing an organ with a perfusion solution comprising A1AT under normothermic temperatures are provided.

“Organ,” as used herein, refers to a whole bodily organ from a human or other mammal or a section of such a bodily organ, wherein the section of an organ is capable of being transplanted into a human or other mammal recipient so as to at least partially replace function of a diseased or missing organ in the recipient. In some embodiments, the organ is a lung, heart, liver, kidney, pancreas, small bowel, or other transplantable organ. In some embodiments, the lung is a whole lung. In some embodiments, the lung is a lobe of a lung. In some embodiments, the lung is a section of a lung. In some embodiments, the liver is a whole liver. In some embodiments, the liver is a section of a liver. In some embodiments, the pancreas is a whole pancreas. In some embodiments, the pancreas is a section of a pancreas. For example, in some embodiments the section of pancreas is a section of pancreatic tissue comprising pancreatic islet cells. In some embodiments, the small bowel is a whole small bowel. In some embodiments, the small bowel is a section of a small bowel.

“Ex vivo organ perfusion,” as used herein, refers to the perfusion of an organ in an ex vivo environment, i.e., external to the body, by methods understood in the art.

“Ex vivo lung perfusion” or “EVLP,” as used herein, refers to the perfusion of a lung in an ex vivo environment, i.e. external to the body, by methods understood in the art. The lung may be a human lung or an animal lung, such as a porcine lung. The lung may be a whole lung, a lobe of a lung, or a section of a lung.

In some embodiments, a method of ex vivo organ perfusion comprises perfusing an organ with a perfusion solution comprising A1AT under normothermic temperatures. In some embodiments, a method of ex vivo organ perfusion comprises perfusing a lung, a heart, a liver, a kidney, a pancreas, or a small bowel with a perfusion solution comprising A1AT under normothermic temperatures. In some embodiments, a method of ex vivo lung perfusion comprises perfusing a lung with a perfusion solution comprising A1AT using the Toronto technique, as described in Cypel, M., et al., Technique for prolonged normothermic ex vivo lung perfusion. J. Heart Lung Transplant, 2008. 27(12): p. 1319-25.

In some embodiments, a donor organ is connected to a perfusion system comprising a platform to place the organ, a vessel containing a perfusion solution comprising A1AT as described herein, a pump, and a circuit (e.g., for transferring a perfusion solution to the organ and collecting the perfusate from the organ). In some embodiments, the perfusion system is a closed system. In some embodiments, the perfusion system is a partially-closed system wherein some components are contained in a housing. In some embodiments, a perfusion system comprises a reservoir containing a heat-transfer fluid (e.g., water) for heat exchange with a vessel comprising a perfusion solution as described herein. In some embodiments, a perfusion system comprises a heater/cooler for maintaining the temperature of the vessel, the fluid in the reservoir, the temperature of the circuit, the temperature of the closed system, or the temperature of the perfusion solution as described herein.

In some embodiments, a donor lung is connected to a perfusion system comprising a platform to place the lung, a vessel containing a perfusion solution comprising A1AT as described herein, a pump, a ventilator, a perfusate gas monitor, an oxygen supply, and a circuit.

A donor organ may be perfused with a perfusion solution comprising A1AT as described herein. A perfusion solution as described herein, a vessel for containing a perfusion solution, a heat-exchange reservoir, a heat-transfer fluid (e.g., water), or a circuit may have a normothermic temperature of from 30° C. to 38° C., of from 32° C. to 38° C., of from 34° C. to 38° C., of from 36° C. to 38° C., of 30° C., of 31° C., of 32° C., of 33° C., of 34° C., of 35° C., of 36° C., of 37° C., or of 38° C.

In some embodiments, a method of ex vivo organ perfusion comprises perfusing an organ with a perfusion solution comprising A1AT under normothermic temperatures and physiological perfusion pressure. For example, a perfusion solution comprising A1AT may be run through a pump before entering the donor organ.

In some embodiments, a method of ex vivo lung perfusion comprises perfusing a lung with a perfusion solution comprising A1AT under normothermic temperatures and ventilating the lung. In some embodiments, the lung is ventilated with a gas having an oxygen content of from 21% to 100% (equivalent to 0.21 to 1.0 FiO₂ (fraction of inspired oxygen)). In some embodiments, the lung is ventilated with gas having an oxygen content of from 20% (0.2 FiO₂) to 100% (1.0 FiO₂), from 25% (0.25 FiO₂) to 90% (0.9 FiO₂), from 30% (0.3 FiO₂) to 80% (0.8 FiO₂), from 40% (0.4 FiO₂) to 70% (0.7 FiO₂), from 50% (0.5 FiO₂) to 60% (0.6 FiO₂). In some embodiments, the lung is ventilated with gas comprising having an oxygen content of 21% (0.21 FiO₂). In some embodiments, the lung is ventilated with a gas having an oxygen content of 100% (1.0 FiO₂).

In some embodiments, a donor organ is perfused for a period of time of from 1 hour to 6 hours, of from 1 hour to 12 hours, of from 1 hour to 13 hours, of from 1 hour to 14 hours, of from 1 hour to 15 hours, of from 1 hour to 16 hours, of from 1 hour to 18 hours, of from 1 hour to 20 hours, of from 1 hour to 24 hours, of from 1 hour to 36 hours, of from 1 hour to 48 hours, of from 1 hour to 60 hours, of from 1 hour to 72 hours, of at least 4 hours, of at least 6 hours, of at least 7 hours, of at least 8 hours, of at least 8 hours, of at least 10 hours, of at least 11 hours, of at least 12 hours, of from 6 hours to 12 hours, of from 6 hours to 13 hours, of from 6 hours to 14 hours, of from 6 hours to 15 hours, of from 6 hours to 16 hours, of from 6 hours to 18 hours, of from 6 hours to 20 hours, of from 6 hours to 24 hours, of from 6 hours to 36 hours, of from 6 hours to 48 hours, of from 6 hours to 60 hours, of from 6 hours to 72 hours, of from 12 hours to 24 hours, of from 12 hours to 36 hours, of from 12 hours to 48 hours, of from 12 hours to 60 hours, of from 12 hours to 72 hours, of from 24 hours to 36 hours, of from 24 hours to 42 hours, of from 24 hours to 60 hours, of from 24 hours to 72 hours, of from 36 to 42 hours, of from 36 to 48 hours, of from 36 to 60 hours, of 1 hour, of 2 hours, of 3 hours, of 4 hours, of 5 hours, of 6 hours, of 10 hours, of 12 hours, of 15 hours, of 18 hours, of 20 hours, of 24 hours, of 30 hours, of 36 hours, of 42 hours, of 48 hours, of 54 hours, of 60 hours, of 66 hours, of 72 hours, at least 1 day, at least 2 days, at least 3 days, at least 4 days, at least 5 days, at least 6 days, at least 7 days.

In some embodiments, an organ of a deceased donor patient may be initially cooled to a temperature of 4° C. to 12° C. In some embodiments, the organ is treated with a cold Perfadex® solution. This procedure may be performed while the organ is still present in the donor body or after procurement from the donor.

In some embodiments, before perfusing a donor organ with a perfusion solution comprising A1AT, the donor organ is processed, preserved, or stored in a below room temperature environment for a period of time. In some embodiments, before perfusing a donor organ with a perfusion solution comprising A1AT, the donor organ is processed, preserved, or stored in an environment having a temperature of 4° C. to 12° C., of from 4° C. to 8° C., of 4° C. to 10° C., of 4° C., of 5° C., of 6° C., of 7° C. of 8° C., of 9° C., of 10° C., of 11° C., or of 12° C. for a period of time.

In some embodiments, before perfusing a donor organ with a perfusion solution comprising A1AT, the donor organ is processed, preserved, or stored for a period of time of from 1 hour to 6 hours, of from 1 hour to 12 hours, of from 1 hour to 24 hours, of from 1 hour to 36 hours, of from 1 hour to 48 hours, of from 1 hour to 60 hours, of from 1 hour to 72 hours, of from 6 hours to 12 hours, of from 6 hours to 24 hours, of from 6 hours to 36 hours, of from 6 hours to 48 hours, of from 6 hours to 6 hours, of from 6 hours to 72 hours, of from 12 hours to 24 hours, of from 12 hours to 36 hours, of from 12 hours to 48 hours, of from 12 hours to 60 hours, of from 12 hours to 72 hours, of from 24 hours to 36 hours, of from 24 hours to 42 hours, of from 24 hours to 60 hours, of from 24 hours to 72 hours, of from 36 to 42 hours, of from 36 to 48 hours, or of from 36 to 60 hours.

For example, in some embodiments, before perfusing a donor organ with a perfusion solution comprising A1AT, the donor organ is stored in a below room temperature environment (e.g., an environment having a temperature of 4° C.) for a period of time of 1 hour, 2 hours, 3 hours, 4 hours, 5 hours, 6 hours, 10 hours, 12 hours, 15 hours, 18 hours, 20 hours, 24 hours, 30 hours, 36 hours, 42 hours, 48 hours, 54 hours, 60 hours, 66 hours, or 72 hours.

In some embodiments, an organ of a deceased donor patient may be perfused ex vivo at a normothermic temperature as described herein and then cooled to a temperature of 4° C. to 12° C. In some embodiments, the organ is treated with a cold Perfadex® solution.

In some embodiments, after perfusing a donor organ with a perfusion solution comprising A1AT, for example at a normothermic temperature, the donor organ is processed, preserved, or stored in a below room temperature environment for a period of time. In some embodiments, after perfusing a donor organ with a perfusion solution comprising A1AT, for example at a normothermic temperature, the donor organ is processed, preserved, or stored in an environment having at a temperature of 4° C. to 12° C., of from 4° C. to 8° C., of 4° C. to 10° C., of 4° C., of 5° C., of 6° C., of 7° C. of 8° C., of 9° C., of 10° C., of 11° C., or of 12° C. for a period of time.

In some embodiments, after perfusing a donor organ with a perfusion solution comprising A1AT at a normothermic temperature, the donor organ is processed, preserved, or stored for a period of time of from 1 hour to 6 hours, of from 1 hour to 12 hours, of from 1 hour to 24 hours, of from 1 hour to 36 hours, of from 1 hour to 48 hours, of from 1 hour to 60 hours, of from 1 hour to 72 hours, of from 6 hours to 12 hours, of from 6 hours to 24 hours, of from 6 hours to 36 hours, of from 6 hours to 48 hours, of from 6 hours to 6 hours, of from 6 hours to 72 hours, of from 12 hours to 24 hours, of from 12 hours to 36 hours, of from 12 hours to 48 hours, of from 12 hours to 60 hours, of from 12 hours to 72 hours, of from 24 hours to 36 hours, of from 24 hours to 42 hours, of from 24 hours to 60 hours, of from 24 hours to 72 hours, of from 36 to 42 hours, of from 36 to 48 hours, or of from 36 to 60 hours.

For example, in some embodiments, after perfusing a donor organ with a perfusion solution comprising A1AT, for example at a normothermic temperature, the donor organ is processed, preserved, or stored in a below room temperature environment (e.g., an environment having a temperature of 4° C.) for a period of time of 1 hour, 2 hours, 3 hours, 4 hours, 5 hours, 6 hours, 10 hours, 12 hours, 15 hours, 18 hours, 20 hours, 24 hours, 30 hours, 36 hours, 42 hours, 48 hours, 54 hours, 60 hours, 66 hours, or 72 hours.

In some embodiments, a donor organ is evaluated before, during, at the termination of, or after the ex vivo perfusion procedure. Whether a donor organ is acceptable for transplantation may be determined based on at least one physiological function, biological marker, or another attribute of the donor organ. If acceptable, an ex vivo perfused donor organ may be transplanted into a recipient.

In some embodiments, at least one physiological function, biological marker, or another attribute of the donor organ is measured before the ex vivo perfusion, during the ex vivo perfusion, at the termination of the ex vivo perfusion, or at a time after the ex vivo perfusion. In some embodiments, the physiological function of the donor organ is increased by an ex vivo organ perfusion procedure, or the level of a biological marker or other attribute of the donor organ is improved. In some embodiments, the physiological function, biological marker, or other attribute of the donor organ at the termination of or at a time after the ex vivo perfusion is increased compared to the physiological function, the biological marker, or other attribute at a time before or during the ex vivo perfusion. In some embodiments, the physiological function, biological marker, or other attribute of the donor organ is decreased by an ex vivo organ perfusion procedure. In some embodiments, the physiological function, biological marker, or other attribute of the donor organ at the termination of or at a time after the ex vivo perfusion is decreased compared to the physiological function, the biological marker, or other attribute at a time before or during the ex vivo perfusion.

In some embodiments, before, during, at the termination of, or after an ex vivo lung perfusion procedure, pulmonary artery (PA) oxygen pressure (PO₂), left atrial (LA) PO₂, difference in PO₂ between the LA and PA (Delta PO₂), pulmonary vascular resistance (PVR), peak inspiratory pressure (Ppeak), plateau airway pressure (Pplat), dynamic pulmonary compliance (Cdyn), static pulmonary compliance (Cstat), or ratio of arterial oxygen partial pressure to fractional inspired oxygen (PaO₂/FiO₂) may be measured or determined. Whether a donor lung is acceptable for transplantation may be determined based on at least one physiological function, biological marker, or another attribute of the donor lung, such as those described above. If acceptable, an ex vivo perfused donor lung may be transplanted into a recipient

In some embodiments, the pulmonary arterial pressure (PAP) at the termination of the ex vivo perfusion or at a time after the ex vivo perfusion is less than the PAP at a time before or during the ex vivo perfusion. In some embodiments, the pulmonary vascular resistance (PVR) at the termination of the ex vivo perfusion or at a time after the ex vivo perfusion is less than the PVR at a time before or during the ex vivo perfusion. In some embodiments, the peak inspiratory pressure (Ppeak) sampled at the termination of the ex vivo perfusion or at a time after the ex vivo perfusion is less than the Ppeak sampled at a time before or during the ex vivo perfusion. In some embodiments, the plateau airway pressure (Pplat) sampled at the termination of the ex vivo perfusion or at a time after the ex vivo perfusion is less than the Pplat sampled at a time before or during the ex vivo perfusion.

In some embodiments, the dynamic pulmonary compliance (Cdyn) of perfusate sampled at the termination of the ex vivo perfusion or at a time after the ex vivo perfusion is (a) greater than the Cdyn sampled at a time before or during the ex vivo perfusion, or (b) maintained compared to the Cdyn sampled at a time before or during the ex vivo perfusion. In some embodiments, the static pulmonary compliance (Cstat) sampled at the termination of the ex vivo perfusion or at a time after the ex vivo perfusion is (a) greater than the Cstat sampled at a time before or during the ex vivo perfusion, or (b) maintained compared to the Cstat sampled at a time before or during the ex vivo perfusion.

In some embodiments, the ratio of arterial oxygen partial pressure to fractional inspired oxygen (PaO₂/FiO₂) at the termination of the ex vivo perfusion or at a time after the ex vivo perfusion is (a) greater than the PaO₂/FiO₂ at a time before or during the ex vivo perfusion, or (b) maintained compared to the PaO₂/FiO₂ at a time before or during the ex vivo perfusion. In some embodiments, the PaO₂/FiO₂ before the ex vivo perfusion is less than 250 mmHg, less than 275 mmHg, less than 300 mmHg, less than 325 mmHg, less than 350 mmHg, or less than 400 mmHg. In some embodiments, the PaO₂/FiO₂ during the ex vivo perfusion, at the termination of the ex vivo perfusion, or at a time after the ex vivo perfusion is at least 300 mmHg or greater, at least 325 mmHg or greater, at least 350 mmHg or greater, at least 375 mmHg or greater, at least 400 mmHg or greater, at least 425 mmHg or greater, at least 450 mmHg or greater, at least 475 mmHg or greater, or at least 500 mmHg or greater. In some embodiments, PaO₂/FiO₂ is measured by the oxygen pressure (PO₂) in the left atrium.

In some embodiments, the difference in oxygen pressure (PO₂) between the left atrium (LA) and pulmonary artery (PA) (Delta PO₂) at the termination of the ex vivo perfusion or at a time after the ex vivo perfusion is (a) greater than the Delta PO₂ at a time before or during the ex vivo perfusion, or (b) maintained compared to the Delta PO₂ at a time before or during the ex vivo perfusion.

To “increase” means to increase, improve, or augment an activity, function, or amount as compared to a reference.

In some embodiments, by “increase” is meant the ability to cause an overall increase of 5% or greater, of 10% or greater, of 20% or greater, of 30% or greater, of 40% or greater, of 50% or greater, of 60% or greater, of 70% or greater, of 80% or greater, of 90% or greater, of 100% or greater relative to a reference value. In some embodiments, by “increase” is meant the ability to cause an overall increase of 5% to 50%, of 10% to 20% of 50% to 100%, of 25% to 70% relative to a reference value. In some embodiments, by “increase” is meant the ability to cause an overall increase of 5%, 10%, 15%, 20%, 25%, 30%, 35%, 40%, 45%, 50%, 55%, 60%, 65%, 70%, 75%, 80% 85%, 90%, 95%, or greater.

To “decrease” means to decrease, reduce, or arrest an activity, function, or amount as compared to a reference.

In some embodiments, by “decrease” is meant the ability to cause an overall decrease of 5% or greater, of 10% or greater, of 20% or greater, of 30% or greater, of 40% or greater, of 50% or greater, of 60% or greater, of 70% or greater, of 80% or greater, of 90% or greater, of 100% or greater relative to a reference value. In some embodiments, by “decrease” is meant the ability to cause an overall decrease of 5% to 50%, of 10% to 20% of 50% to 100%, of 25% to 70% relative to a reference value. In some embodiments, by “decrease” is meant the ability to cause an overall decrease of 5%, 10%, 15%, 20%, 25%, 30%, 35%, 40%, 45%, 50%, 55%, 60%, 65%, 70%, 75%, 80% 85%, 90%, 95%, or greater.

To “maintain” means an activity, function, or amount as compared to a reference is maintained at a similar value or level.

In some embodiments, by “maintain” is meant within 0.5% above, 0.5% below, 1% above, 1% below, 2% above, 2% below, 3% above, 3% below, 4% above, 4% below, 5% above, 5% below, 10% above, 10% below, 15% above, 15% below, 20% above, or 20% below a reference value. In some embodiments, by “maintain” is meant within 20% or less above, 20% or less below, 15% or less above, 15% or less below, 10% or less above, 10% or less below, 5% or less above, 5% or less below, 1% or less above, or 1% or less below a reference value.

A “reference” as used herein, refers to any sample, standard, or level that is used for comparison purposes.

In some embodiments, an amount is increased, decreased, or maintained over a period of time, relative to a control organ over the same period of time. In some embodiments, an amount is increased, decreased, or maintained over a period of time, relative to an amount at an earlier point in time for the same organ.

### Transplantation of Organs Ex Vivo Perfused with A1AT-Containing Perfusion Solutions

Methods of transplanting an organ that has been ex vivo perfused with a perfusion solution comprising A1AT are also provided. In some embodiments, a method of transplanting an organ comprises perfusing an organ ex vivo with a perfusion solution comprising A1AT as provided herein and transplanting the ex vivo perfused organ into a recipient. In some embodiments, the perfusion solution is exposed to an environment having a normothermic temperature.

In some embodiments, in addition to the use of A1AT during ex vivo perfusion of the organ, A1AT is also administered to the organ recipient before, during, or after the organ is transplanted. In some embodiments, A1AT is administered to the organ recipient before the organ transplant. In some embodiments, A1AT is administered to the organ recipient during the organ transplant. In some embodiments, A1AT is administered to the organ recipient after the organ is transplanted. In some embodiments, the A1AT is administered to the organ recipient intravenously or subcutaneously. In some embodiments, the agent is administered to the organ recipient by intravenous injection or subcutaneous injection, etc.).

In some embodiments, at least one additional therapeutic agent other than A1AT is administered to the organ recipient before, during, or after the organ is transplanted. For example, in some recipients, it may be desirable to add agents that may reduce inflammatory and/or immunological symptoms associated with the transplantation of an exogenous organ. In some embodiments, the agent is an anti-inflammatory agent. In some embodiments, the agent is an immunomodulatory agent. In some embodiments, the agent is interferon, an interferon derivative (e.g., betaseron, beta-interferon, etc.), a prostane derivative (e.g., iloprost, cicaprost, etc.), a glucocorticoid (e.g., cortisol, prednisolone, methyl-prednisolone, dexamethasone, etc.), an immunosuppressive (e.g., cyclosporine A, FK-506, methoxsalene, thalidomide, sulfasalazine, azathioprine, methotrexate, etc.), a lipoxygenase inhibitor (e.g., zileutone, MK-886, WY-50295, SC-45662, SC-41661A, BI-L-357, etc.), a leukotriene antagonist, a peptide derivative (e.g., adrenocorticotropic hormone (ACTH) or an analog, etc.), a soluble TNF-receptor, an anti-TNF antibody, a soluble receptor of an interleukin, an anti-interleukin antibody, an anti-inflammatory cytokine, a pro-inflammatory cytokine antagonist, a T-cell protein, an anti-interleukin receptor antibody, an anti-CD20 antibody (e.g., rituximab, etc.), a C1 esterase inhibitor, calcipotriol, mycophenolate mofetil (such as Cellcept®), a mycophenolate mofetil analog, IdeS (IgG-degrading enzyme of S. pyogenes), an IL6 antagonist (e.g., an anti-IL6R antibody, etc.), IL6R antagonist (e.g., an anti-IL6R antibody, etc.), ann intravenous IgG preparation (IVIG; e.g., Privigen®, Gammagard®, Gamunex®) or subcutaneous IgG preparation (e.g. Hizentra®, HyQvia®), an anti-CD25/IL-2 receptor antibody such as basiliximab, daclizumab, alemtuzumab, and/or an anti-thymocyte globulin (ATG; e.g., Thymoglobulin®, etc.). In some embodiments, the additional therapeutic agent is one or more of C1INH, IVIG, SCIG, an anti-CD25/IL-2 receptor antibody (e.g. basilixumab, daclizumab, or alemtuzumab), and ATG. In some embodiments, both A1 AT and the additional therapeutic agent are administered to the recipient before, during, and/or after transplantation.

In some embodiments, the A1AT and at least one additional agent(s) are administered in combination. Administration “in combination with” one or more further agents includes simultaneous (concurrent), consecutive, or sequential administration, in any order. The term “concurrently” is used herein to refer to administration of two or more agents, where at least part of the administration overlaps in time or where the administration of one agent falls within a short period of time relative to administration of the other therapeutic agent. For example, the two or more agents may be administered with a time separation of no more than about a specified number of minutes, hours, or days. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, the two or more agents may be administered with a time separation of more than about a specified number of minutes. As used herein, “in conjunction with” refers to administration of one treatment modality in addition to another treatment modality. As such, “in conjunction with” refers to administration of one treatment modality before, during, or after administration of the other treatment modality to the subject.

The following examples illustrate particular aspects of the disclosure and are not intended in any way to limit the disclosure.

### EXAMPLES

### Example 1

### Donor Lung Retrieval and Preservation

Lungs from 15 Yorkshire male pigs (approximately 30 kg) were retrieved and preserved at 4° C. for 24 hours. The pigs were sedated with Ketamine (20 mg/kg IM), Midazolam (0.3 mg/kg IM) and Atropine (0.04 mg/kg IM), and then anesthetized with inhaled isoflurane (1-3%), followed by a continuous intravenous infusion of Remifentanyl (9-30 μg/kg/h) during donor retrieval. The animals were intubated and ventilated at an inspired oxygen fraction (FiO₂) of 0.5, a frequency of 15 breaths/min, a positive end-expiratory pressure (PEEP) of 5 cm H₂O, and controlled pressure above PEEP of 15 cm H₂O.

A median sternotomy was performed and sodium heparin (10,000 IUs) were injected systemically. The main pulmonary artery (PA) was cannulated and connected with a flushing line. The superior vena cava and the inferior vena cava were ligated with silk. The left atrial (LA) appendage was transected for drainage and the lungs were flushed through the PA with 60 mL/kg of Perfadex® lung preservation solution at 4° C. from a height of 30 cm above the heart. The heart-lung block was removed with the lung inflated, and an additional 1 L of cold Perfadex® solution was instilled via retrograde flush from LA to PA. Twelve of the 15 donor lungs were immersed/preserved in cold Perfadex® solution and stored at 4° C. for 24 h. Three of the donor lungs were excluded due to technical reasons.

### Example 2

### A1AT Treatment During Ex Vivo Lung Perfusion (EVLP)

The 12 preserved, donor lungs were subjected to EVLP for 12 h using the following procedure. The LA cuff was trimmed and sewn to a cannula with a 4-0 polypropylene running suture. The flush cannula was inserted into the main PA proximal to its bifurcation and secured with two heavy silk ties. With the trachea clamped at the level of the carina, the staple line was opened and a conventional endotracheal tube was inserted and secured with two heavy silk ties. A second retrograde flush with 1 L of Perfadex® solution (XVIVO Perfusion, Goteborg, Sweden) was performed. The inflated lungs were then transferred to the XVIVO chamber (XVIVO, Denver, Colo., USA) and connected to the perfusion circuit according to the procedure described in Nishina, K., et al., ONO-5046, an elastase inhibitor, attenuates endotoxin-induced acute lung injury in rabbits, Anesth Analg, 1997. 84(5): p. 1097-103.

The donor pig lungs were randomly to two groups (n=6 for each group). The treatment group received 3 mg/mL A1AT (Zemaira®, CSL Behring, King of Prussia, Pa., USA) in Steen® solution (XVIVO Perfusion, Goteborg, Sweden) and the control group received Steen® solution only. In the treatment group, A1AT was freshly added to Steen® solution prior to the procedure. The perfusate flow was initiated at 10% of the full flow rate at room temperature. The perfusate was actively warmed, and when the temperature of the perfusate reached 32° C., ventilation was started with an FiO₂ of 0.21, tidal volume (V_{T}) of 7 mL/kg, frequency of 7 breaths/min, and PEEP of 5 cm H₂O. The temperature of the perfusate was gradually increased to 37° C. The oxygenated perfusate draining from the lungs was deoxygenized using a membrane oxygenator that was swept with a gas mixture of nitrogen (86%), carbon dioxide (8%), and oxygen (6%). The perfusate was gradually increased to the calculated full flow rate, which was 40% of estimated cardiac output (CO=100 mL/kg). During EVLP, 100 mL of perfusate was replaced every hour with Steen® solution freshly prepared with or without A1AT according to the group.

### Example 3

### Donor Lung Characteristics

Donor body weight, partial pressure of oxygen (PaO2), cold ischemic time (CIT), and dynamic pulmonary compliance of the A1AT and control groups are provided in Table 1.

TABLE 1

| Donor lung and EVLP | Control group | A1AT group |
|---|---|---|
| characteristics | (n = 6) | (n = 6) |
| Donor body weight, kg | 32.00 ± 0.84 | 33.98 ± 0.86 |
| Donor PaO₂, mmHg (FiO2 = 50%) | 262.3 ± 18.99 | 248.2 ± 17.23 |
| Donor lung CIT time, hours | 24.19 ± 0.086 | 24.41 ± 0.17 |
| Donor lung Cdyn, ml/cm H₂O | 28.83 ± 2.482 | 33.83 ± 4.861 |

*All variables are recorded as mean ± standard error of the mean.
*PaO₂, partial pressure of O₂ in arterial blood;
FiO₂, fraction of inspired oxygen;
CIT, cold ischemia time;
Cdyn, dynamic pulmonary compliance.

### Example 4

### Physiological Function of Donor Lungs During EVLP

Physiological functions of the 12 donor lungs were monitored hourly during the 12 hour EVLP procedure. The following physiological parameters of lung function during EVLP were recorded: PA pressure, LA pressure, peak inspiratory pressure (Ppeak), plateau airway pressure (Pplat), and dynamic and static compliances. Static compliance (Cstat) represents pulmonary compliance during periods without gas flow, Cstat=VT/(Pplat-PEEP). Dynamic compliance (Cdyn) represents pulmonary compliance during periods of gas flow, Cdyn=V_{T}/(Ppeak−PEEP). Pulmonary recruitment was performed 30 minutes after each assessment by increasing the V_{T} up to 14 mL/kg with subsequent inspiratory hold maneuvers up to 25 cm H₂O for 3 seconds for 3 times.

The physiological results are reflected in the figures. The data presented herein are expressed as mean±standard error of the mean (SEM). A two-way analysis of variance was used to analyze the data from observations over time. Mann-Whitney was utilized for non-parametric tests. Statistical analyses were performed with GraphPad Prism 7 (Prism 7, La Jolla, Calif., USA). Differences were considered significant when the p-value was ≤0.05.

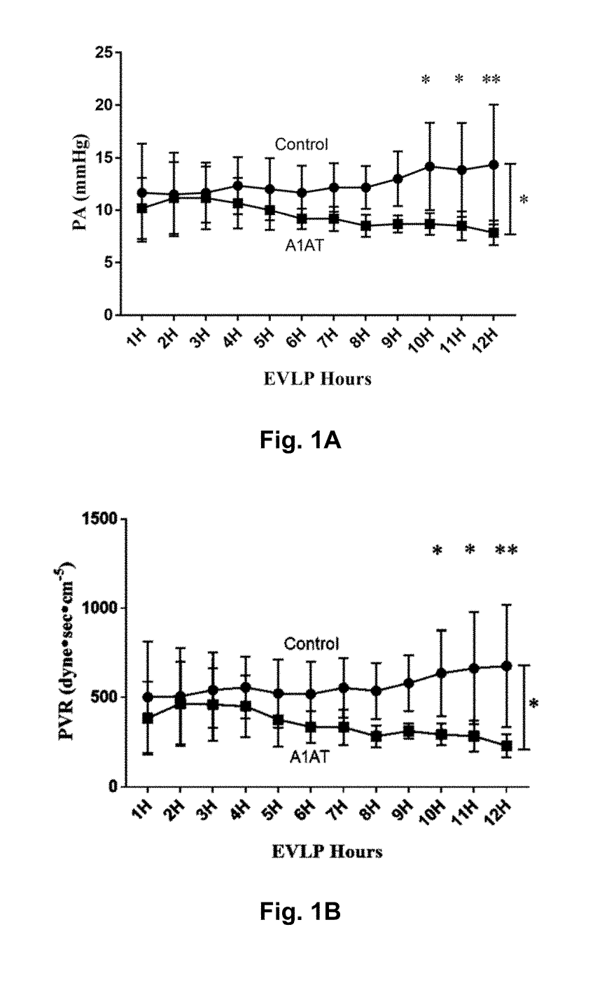

Pulmonary arterial pressure (PAP) (FIG. 1A) and pulmonary vascular resistance (PVR) (FIG. 1B) gradually increased during the EVLP in the control group, while in the treatment group both PAP and PVR remained stable during the first 4 hours and then gradually decreased. Both PAP and PVR were significantly lower in the treatment group at time points of 10 hours (*p<0.05), 11 hours (*p<0.05), and 12 hours of the EVLP (**p<0.01).

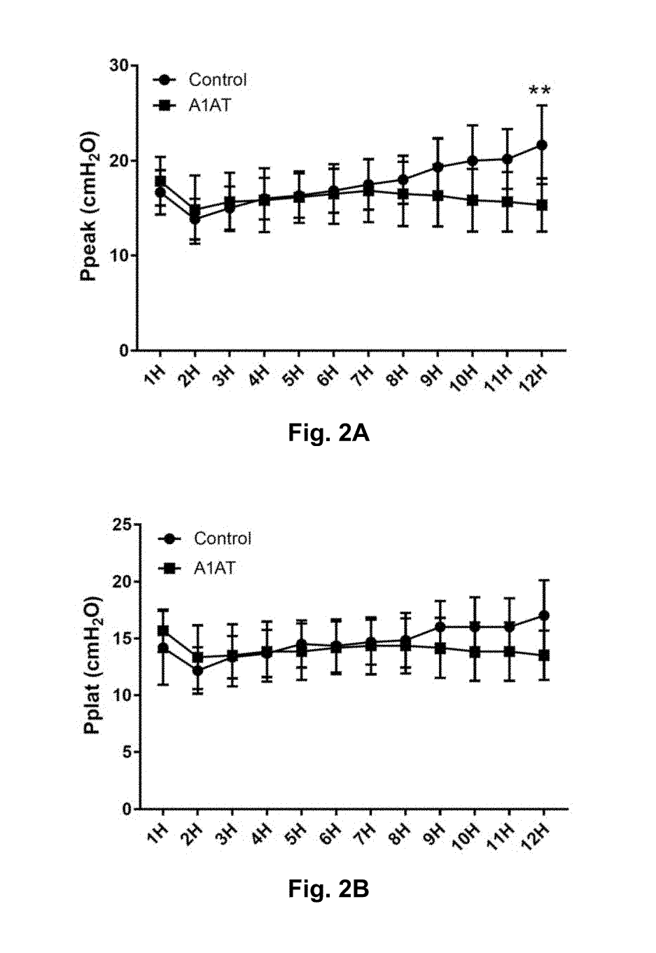

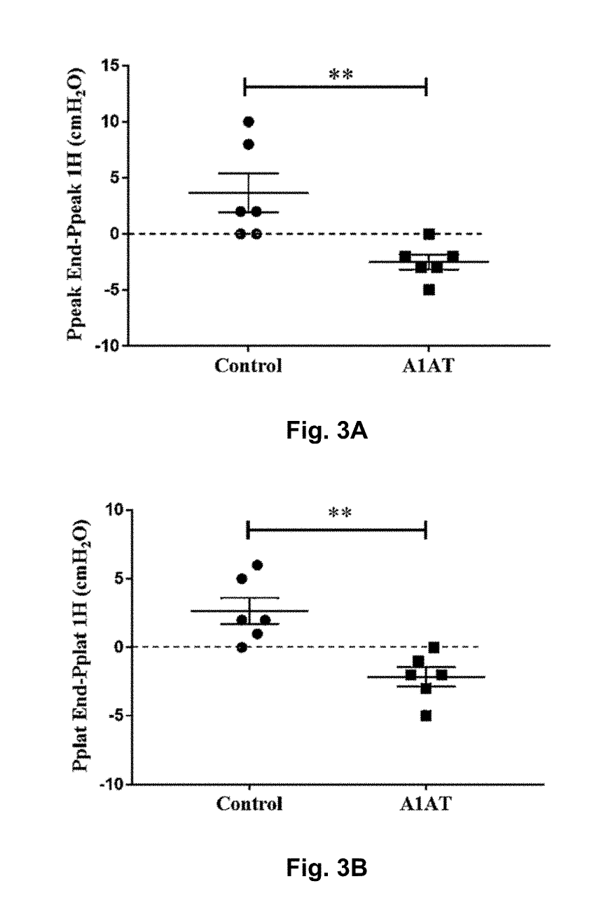

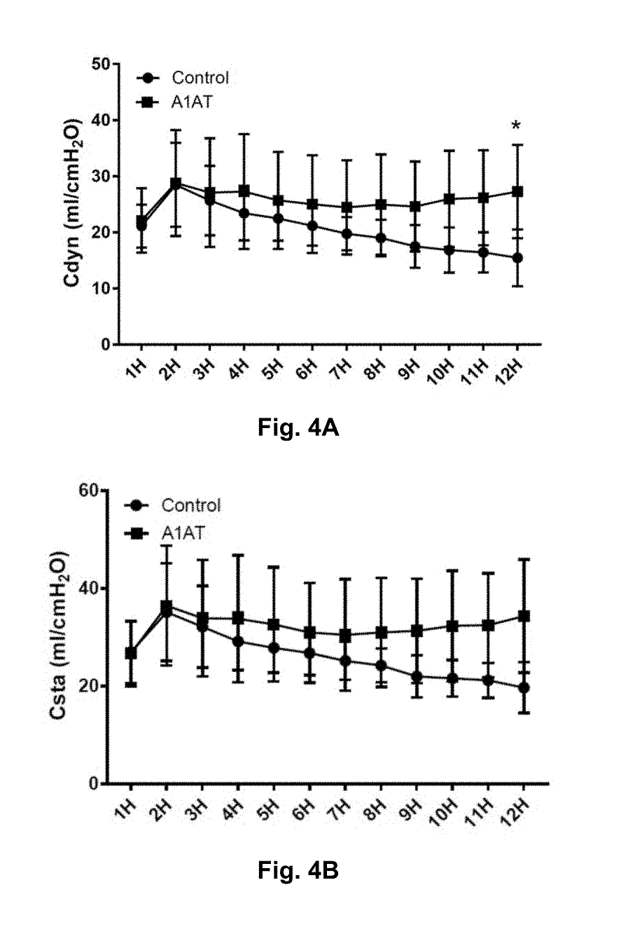

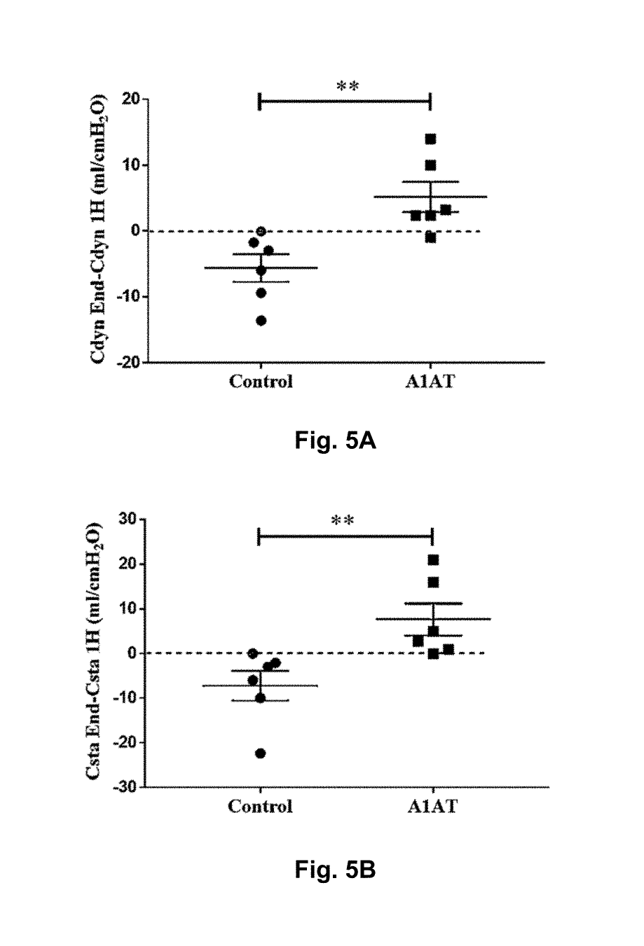

The peak (Ppeak) and plateau (Pplat) airway pressures gradually increased after 7 hours of EVLP in the control group, while they gradually decreased in the A1AT treatment group (FIGS. 2A and 2B). The changes in Ppeak and Pplat from baseline (measured at 1 hour of EVLP) to the end of EVLP were significantly lower (**p<0.01) in the treatment group (FIGS. 3A and 3B). By contrast, the dynamic (Cdyn) and static (Cstat) pulmonary compliances of donor lungs were gradually decreased in the control group, but were maintained at the same levels in the treatment group (FIGS. 4A and 4B). The changes in Cdyn and Cstat from baseline (measured at 1 hour of EVLP) to the end of EVLP were significantly higher (**p<0.01) in the treatment group (FIGS. 5A and 5B).

### Example 5

### Lung Oxygenation During EVLP

Donor lung oxygenation was assessed at 1 hour of EVLP (baseline) and at 12 hours of EVLP. To assess oxygenation, the fraction of inspired oxygen (FiO₂) was increased from 0.21 (equivalent to 21% oxygen like natural air) to 1.0 (100% oxygen) for 5 min. Perfusate samples were taken from the left atrium (LA) and pulmonary artery (PA) for gas analysis. FiO₂ was turn back from 1.0 to 0.21 thereafter.

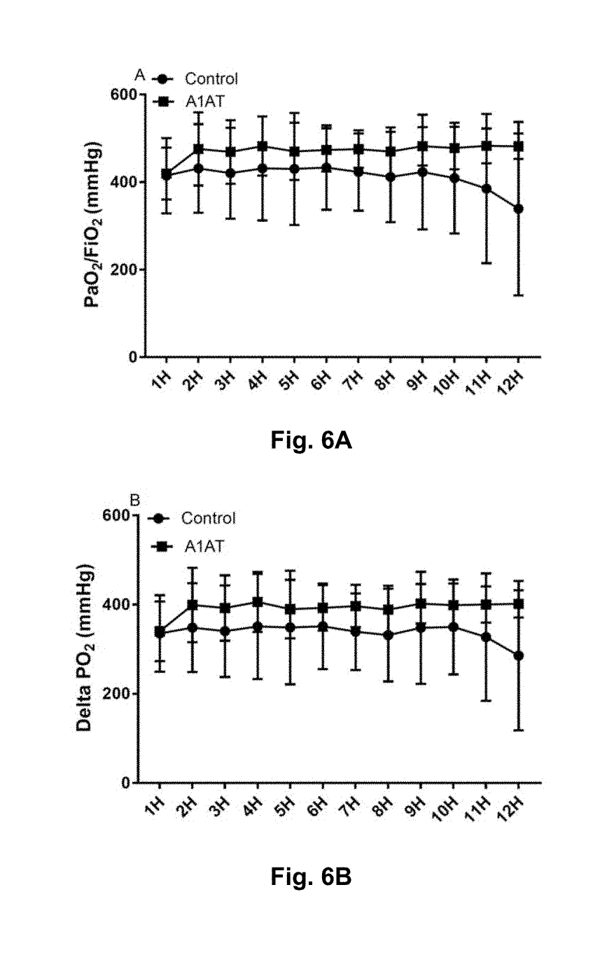

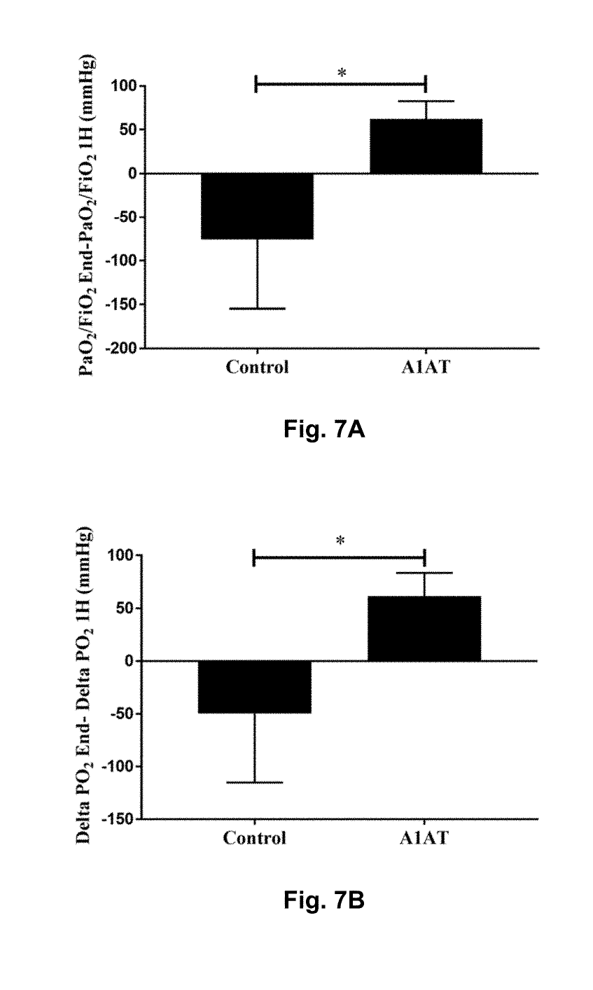

Donor lung oxygenation function was preserved in the A1AT group during EVLP. The partial pressure of oxygen (PO₂) in the LA was measured to reflect the ratio of arterial oxygen partial pressure to fractional inspired oxygen (PaO₂/FiO₂). The PO₂ difference between LA and PA was expressed as Delta PO₂. These values gradually decreased over the 12-hour EVLP period in the control group, while they remained at higher levels in the A1AT treatment group (FIGS. 6A and 6B). Compared with the baseline values measured at 1 hour of EVLP, at the end of 12 hours, PO₂/FiO₂ (FIG. 7A) and Delta PO₂ (FIG. 7B) were decreased in the control group, but increased in the A1AT group. The differences were significantly different between the two groups (*p<0.05).

### Example 6

### Histological Assessment of Acute Lung Injury

At the end of the EVLP procedure, the tissue biopsies were collected from all lobes and were divided into 3 samples: 1) samples kept in −80° C. for subsequent biological assessments, 2) samples fixed in 10% buffered formalin for histological assessment, and 3) samples for measurement of wet-to-dry (W/D) lung weight ratio.

The lung tissue samples fixed in 10% buffered formalin were embedded in paraffin and sectioned onto slides. The slides were stained with hematoxylin and eosin (HE), and evaluated for microscopic lung injury by a pulmonary pathologist in a blinded manner. Both groups showed minor lung injury and no significant difference was noted in HE-stained slides (data not shown).

### Example 7

### Wet-To-Dry Lung Weight Ratio

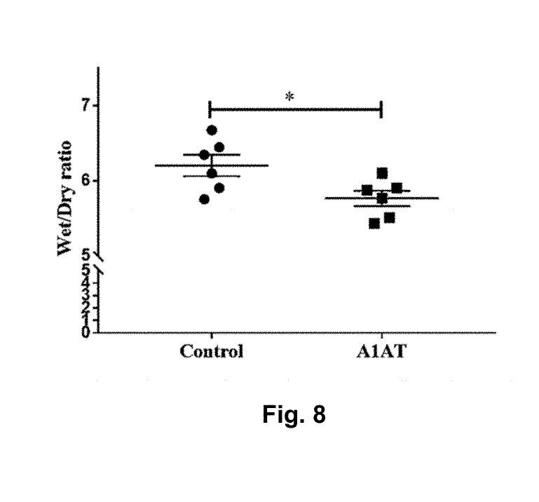

The weight-to-dry (W/D) lung weight ratio, which reflects the extent of pulmonary edema, was calculated by dividing the lung tissue weight before and after drying at 72° C. for 72 hours. At the end of EVLP, the donor lungs' wet-to-dry weight ratio was markedly lower in the A1AT treatment group (5.765±0.1037) than in the control group (6.203±0.1418) (*p<0.05) (FIG. 8).

### Example 8

### A1AT Concentration in Lung Tissue and Perfusate

A1AT concentrations in lung tissue and EVLP perfusate were measured with an ELISA kit specific for human A1AT (SimpleStep ELISA® with SERPINA1 (ab189579, Abcam, Toronto, Canada)) following the manufacturer's instructions. In the treatment group, the concentration of human A1AT in the perfusate was 0.5 mg/mL during the 12 hours of EVLP. Human A1AT was not detected in the control group. The concentration of human A1AT in donor lung tissue of the treatment group at the end of EVLP was 13.49±1.78 μg/mg protein.

### Example 9

### Apoptosis Assessment

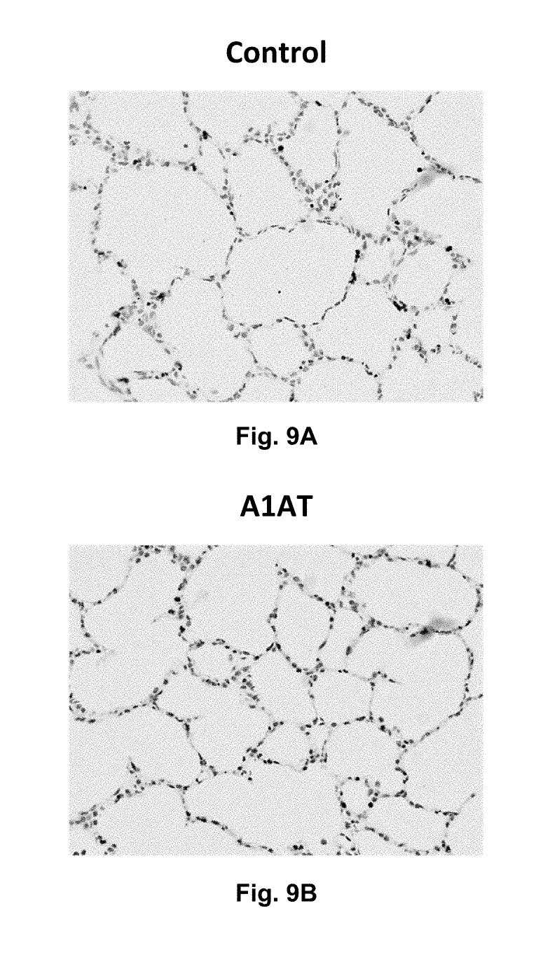

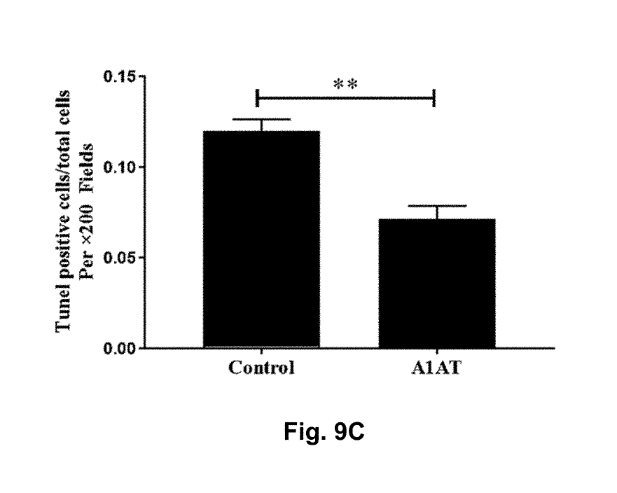

Apoptotic cell death was examined by TUNEL (terminal deoxynucleotidyl transferase dUTP nick-end labeling) staining and quantified in a blinded fashion. See Wijsman, J. H., et al., A new method to detect apoptosis in paraffin sections: in situ end-labeling of fragmented DNA. J Histochem Cytochem, 1993. 41(1): p. 7-12. Using a microscope, 10 fields per slide were photographed. TUNEL-positive brown cells and blue stained nuclei were counted in a blinded manner using NIS-Elements Basic Research Microscope Imaging Software) (Nikon Instruments Inc., USA). Representative TUNEL staining images at 200× magnification from a control sample and a treatment sample at the end of EVLP are shown in FIGS. 9A and 9B, respectively. As shown in FIG. 9A, the alveolar structures in both groups remained intact. The dark brown stained TUNEL positive cells were mainly along the alveolar wall. The ratio of TUNEL-positive (apoptotic) cells to total cells was markedly lower in the A1AT treatment group (7.12±0.75%) than the control group (11.98±0.64%) (**p<0.01) (FIG. 9C).

### Example 10

### Assessment of Inflammatory Mediators

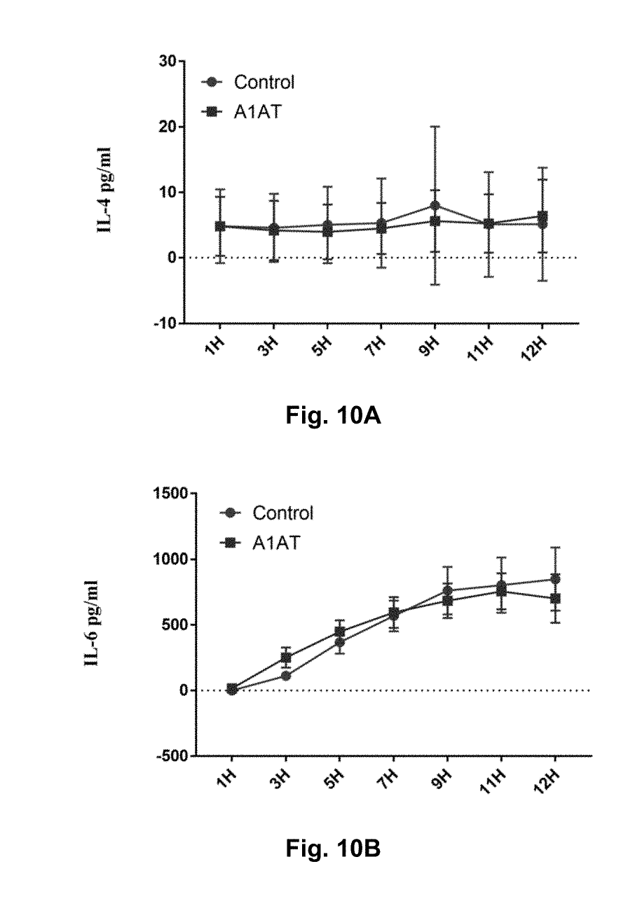

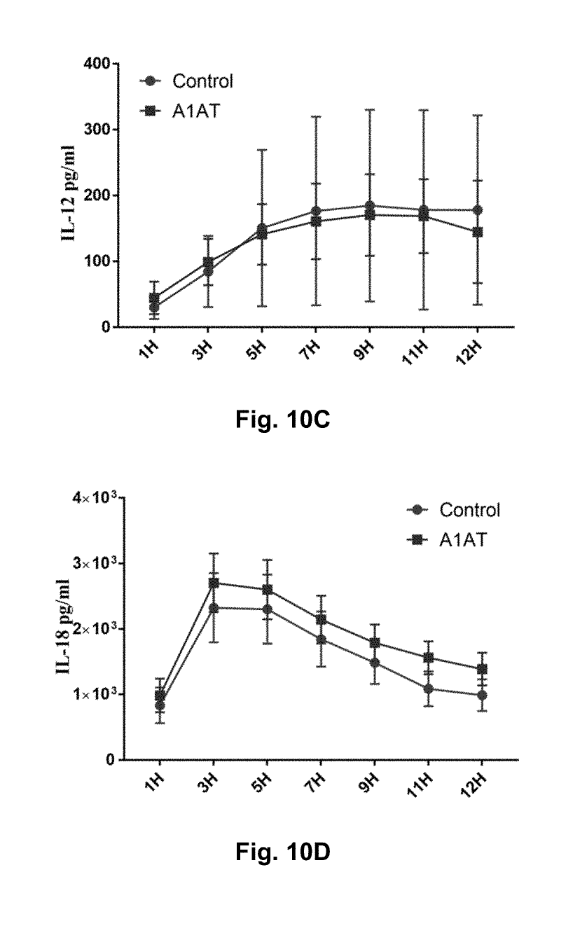

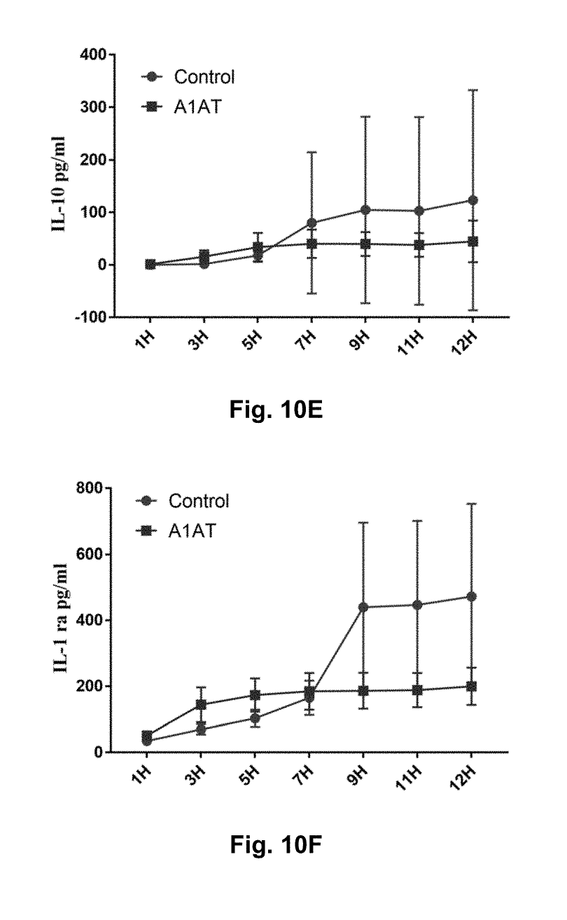

Perfusate samples were collected at each hour of EVLP, aliquoted into 10 tubes, and stored at −80° C. immediately until further analyzed. Pro-inflammatory cytokine levels in the perfusate samples were measured using Miliplex MAP Porcine Cytokine/Chemokine Magnetic Bead Panel 11 (Millipore EMD, Billerica, Mass., USA) for IL-1α, IL-1β, IL-1ra, IL-2, IL-4, IL-6, IL-8, IL-10, IL-12, IL-18, and TNFα. Kits were used according to the manufacturer's instructions, and the analytics were read with a Luminex 100 analyzer (Luminex, Austin, Calif., USA). Data were analyzed with Bio-plex Manager 6.0 (Bio-Rad Laboratories, Mississauga, Canada), and the concentration of each analyte (pg/mL) was plotted versus time (FIGS. 10A-F and 11A-E).

A1AT treatment did not appear to affect the level of IL-4, IL-6, IL-12, IL-18, IL-10, or IL-1 ra when compared to control over the course of the EVLP procedure (FIG. 10A-F). The levels of IL-4 remained unchanged (FIG. 10A), while IL-6 and IL-12 levels continuously increased during the 12 hours of EVLP (FIGS. 10B and 10C). The levels of IL-18 increased during the first 3 hours and then gradually decreased thereafter (FIG. 10D). Two anti-inflammatory cytokines, IL-10 (FIG. 10E) and IL-1 ra (FIG. 10F) gradually increased during EVLP, especially after 7 hours.

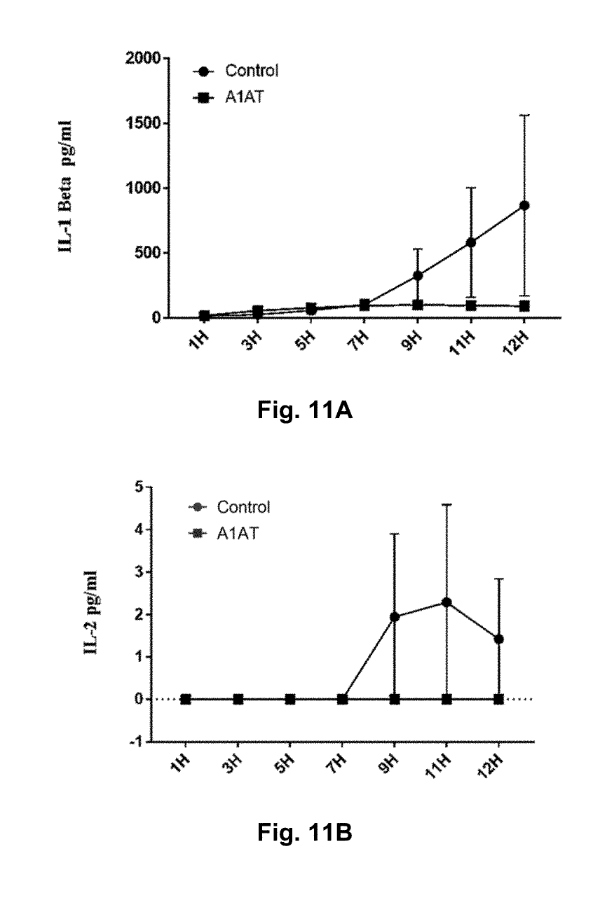

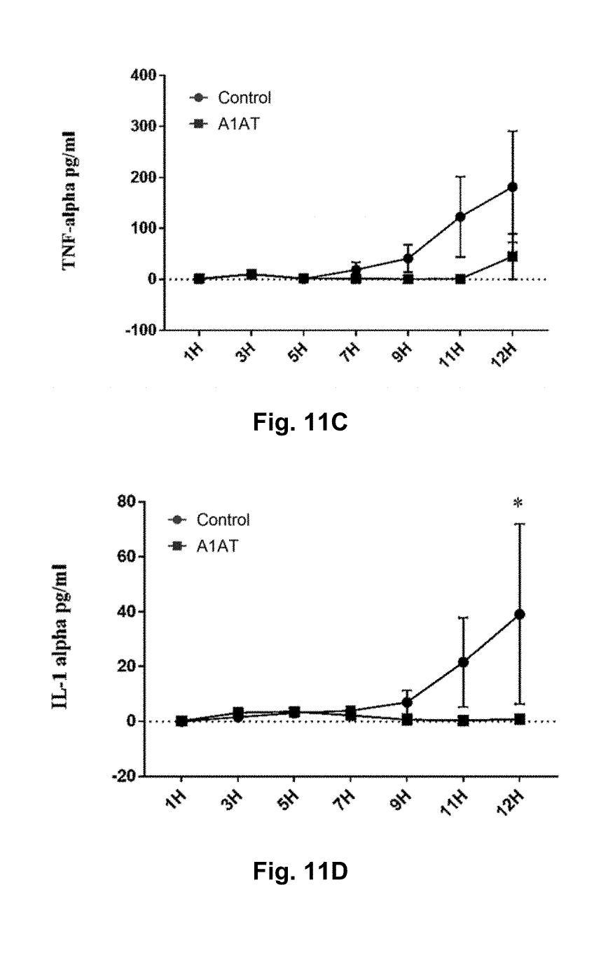

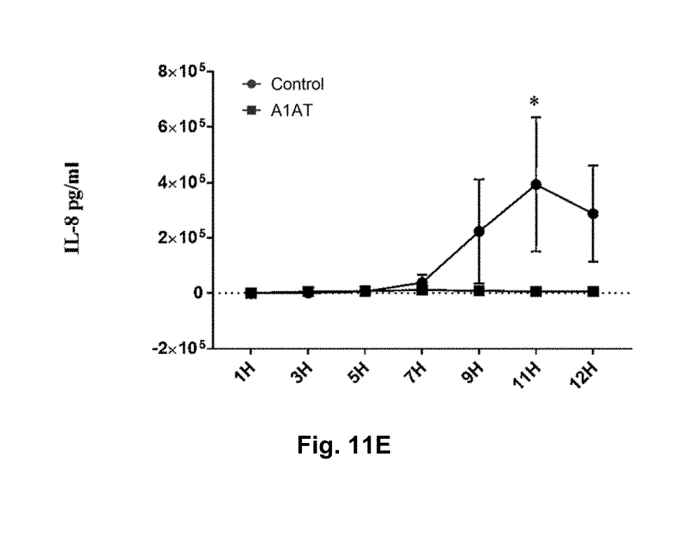

Inflammatory modulators IL-1β (FIG. 11A), IL-2 (FIG. 11B), TNFα (FIG. 11C), IL-1α (FIG. 11D), and IL-8 (FIG. 11E) were almost undetectable in the EVLP perfusate for the first 7 hours, and then gradually increased in the control group. A1AT appeared to block the increase of these cytokines, of which, the levels of IL-1α at 12 hours and IL-8 at 11 hours were significantly different between the two groups (*p<0.05).

### Example 11

### Electrolyte Levels in EVLP Perfusate

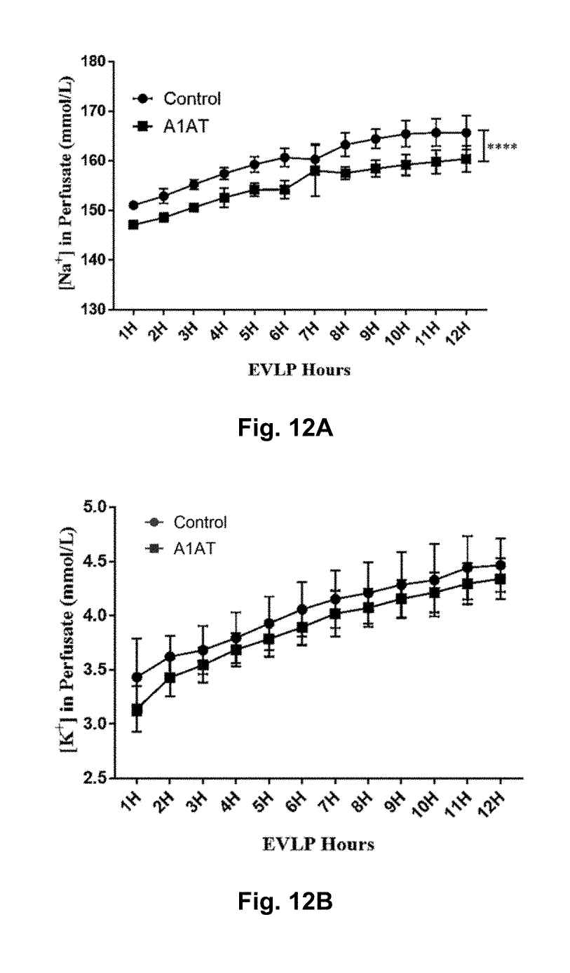

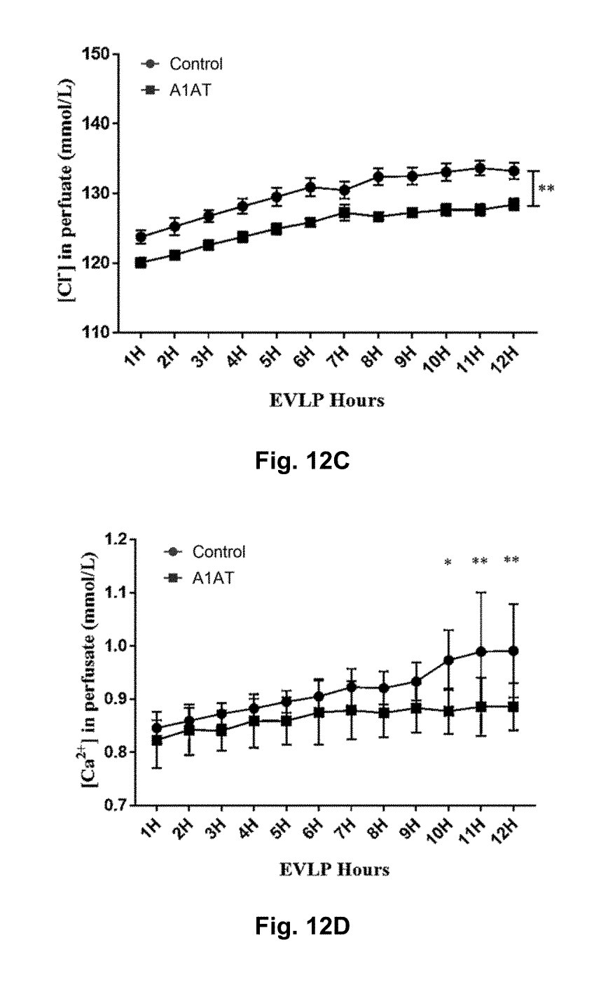

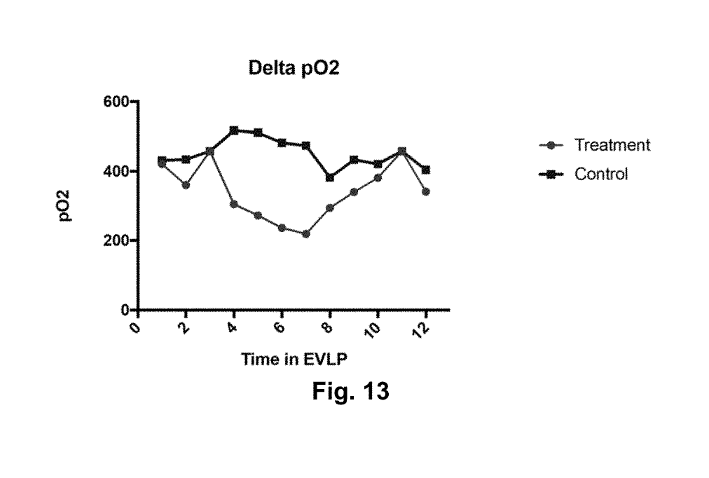

Electrolyte concentrations were measured in EVLP perfusate samples that were collected hourly during the procedure. Sodium (FIG. 12A), potassium (FIG. 12B), chloride (FIG. 12C), and calcium (FIG. 12D) ion concentrations increased over the course of the EVLP procedure in both control and A1AT treatment groups. While no statistically significant difference in potassium ion concentration between the control and treatment groups was detected (FIG. 13B), the sodium ion (FIG. 12A) and chloride ion (FIG. 12C) concentrations were significantly lower in the treatment group during EVLP (****p<0.0001 and **p<0.01, respectively). The calcium ion concentration in perfusate from 10 hours to 12 hours was significantly lower in the A1AT treatment group (FIG. 12D, at 10 hours (*p<0.05), 11 hours (**p<0.01), and 12 hours (**p<0.01)).

### Example 12

### Transplantation of Human Donor Lungs

Human donor lungs are subjected to EVLP under normothermic temperature using Steen® solution containing A1AT at a concentration ranging from 5 mg/mL to 10 mg/mL under ventilation according to the Toronto technique. (See Ref 30.) The human donor lungs may have been or may not have been previously processed, preserved, or stored at 4° C. The EVLP/A1AT-treated donor lungs may be transplanted into a suitable recipient. The recipient may be administered A1AT by intravenous injection prior to or following the transplant procedure.

### Example 13

### Pilot Study of Ex Vivo Perfusion of Human Lungs

Lungs from 5 human multi-organ donors that suffered brain death or cardiac death and that were determined to be clinically unsuitable for transplantation were used for this experiment. The lung block was separated, and right and left lungs were maintained on two separate EVLP circuits for twelve hours at body temperature. Each lung was randomly assigned to either receive A1AT treatment or placebo via the EVLP perfusate.

EVLP was performed immediately after the donor lungs were received by the research laboratory. As per the Toronto EVLP protocol, the lungs were placed in an Xvivo® chamber. A cannula with pressure-monitoring lines was attached to the pulmonary artery (PA) and another to the left atrium (LA). The perfusate was pumped by a centrifugal pump into the oxygenator and heat exchanger, where it was be deoxygenated by a gas mixture (86% N2, 8% CO2, and 6% O2) and heated to normothermia (37° C.) within the first hour.

The EVLP circuit was primed with 1.5 ml of Steen® solution. A1AT or saline placebo was added to the perfusate. 10 mg/ml of A1AT was administered. Normal human A1AT blood levels are 0.9-1.75 mg/ml. A1AT is an acute phase protein that can increase up to four times during an inflammatory response.

Physiological lung function was evaluated hourly for 12 hours. PA pressure, LA pressure, peak airway pressure, plateau pressure, and dynamic and static compliance were recorded and analyzed. Perfusate gas analysis was performed in samples taken from the venous and arterial sides.

The concentration of A1AT in perfusate was measured hourly and the concentration of A1AT was measured in lung tissue at the beginning of EVLP and every three hours until end of EVLP. As previous pig studies showed that more A1AT is consumed in damaged lungs than healthy lungs, it was hypothesized that this can be the result of the protein binding to the neutrophil elastase (NE).

The study also tested safety by evaluating hourly physiologic lung parameters as well as metabolic values in perfusate (ph, glucose, lactate) from the treated lung. The values obtained were compared to the basal values and to the contralateral non treated lung. No deterioration of the parameters in the treated group was seen that correlated with the A1AT levels.

Lung biopsies were taken prior to starting EVLP and every 3 hours during EVLP until the end of 12 hours or as long as the lung was on the circuit. Samples are currently being evaluated to measure the level of apoptosis by using cleaved caspase-3 and TUNEL immunohistochemistry staining. Cytokine levels were measured hurly in perfusate and will be measured in tissue homogenates.

Perfusate samples were taken hourly to test for cytokines and other inflammation markers. Adhesion molecules on the vascular endothelium surface are markers of endothelial activation. They play an important role in leukocyte-endothelial adhesion in the inflammatory response. These adhesion molecules are upregulated on the membrane of activated endothelium in response to stimulation by inflammatory cytokines or endotoxins. A1AT could have an impact on them by directly blocking cytokine release or by binding to IL-8 and or TNF alpha. Soluble intercellular adhesion molecule 1 (sICAM-1), soluble VCAM-1 (sVCAM-1), and soluble E selectin (sE-selectin) levels will be measured in perfusate of EVLP.

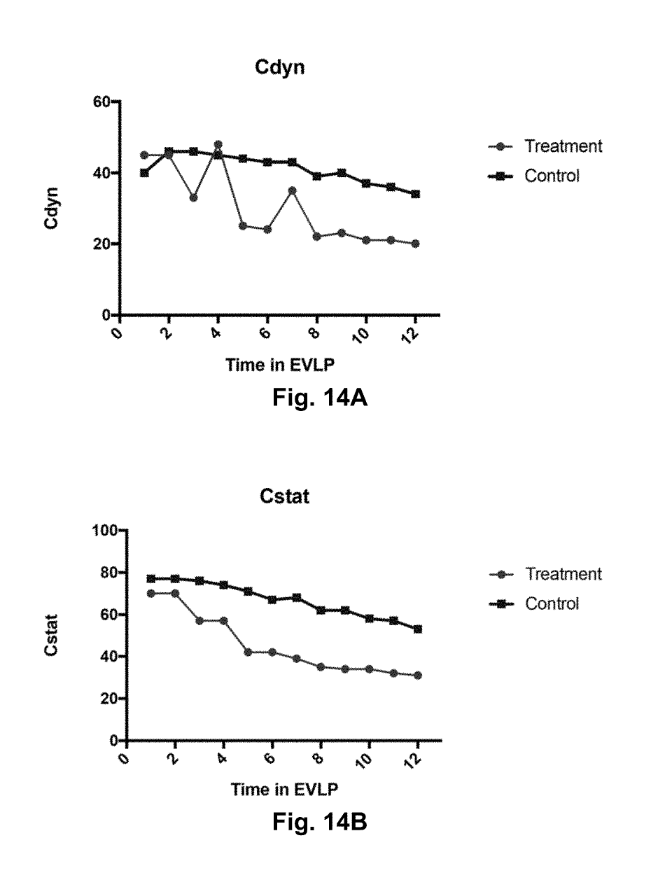

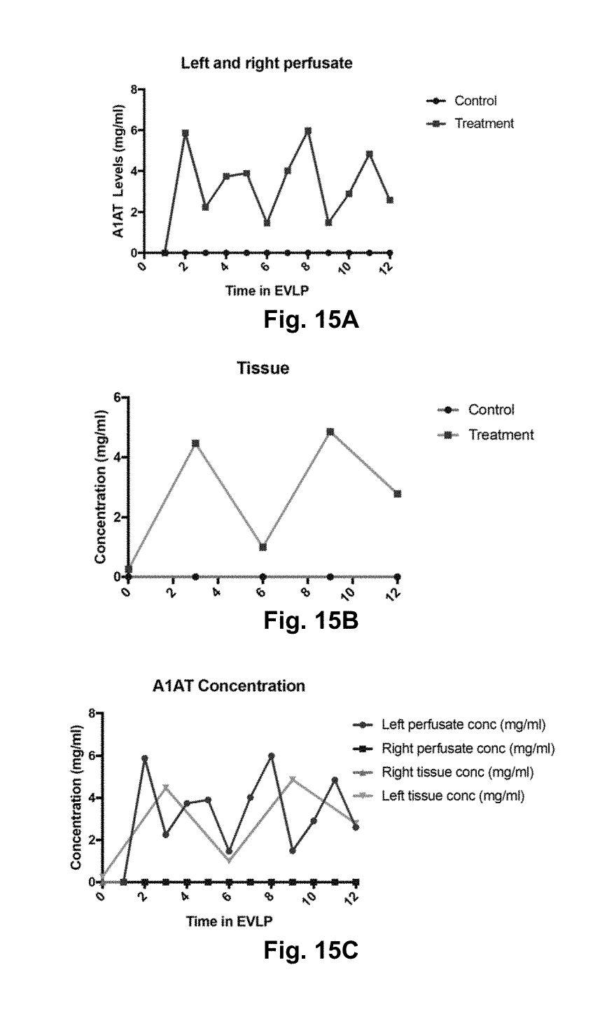

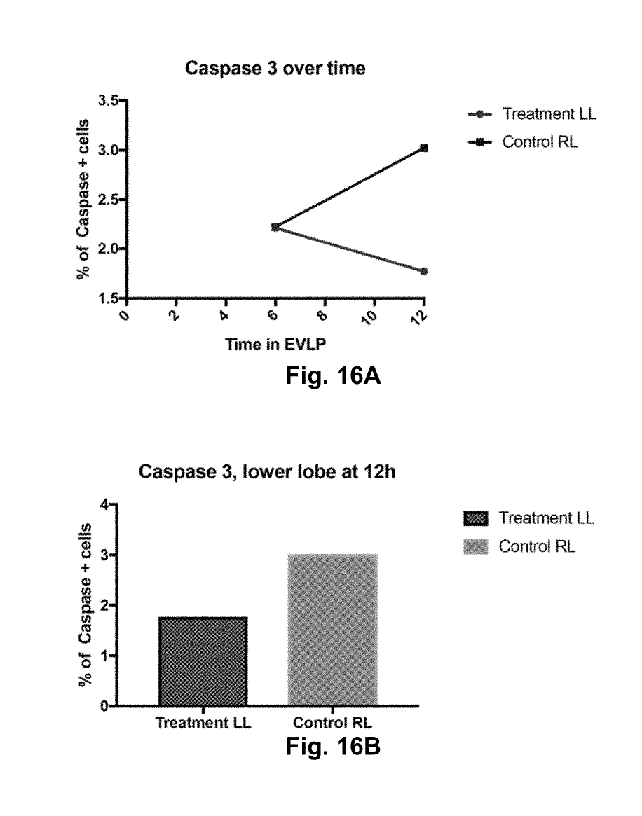

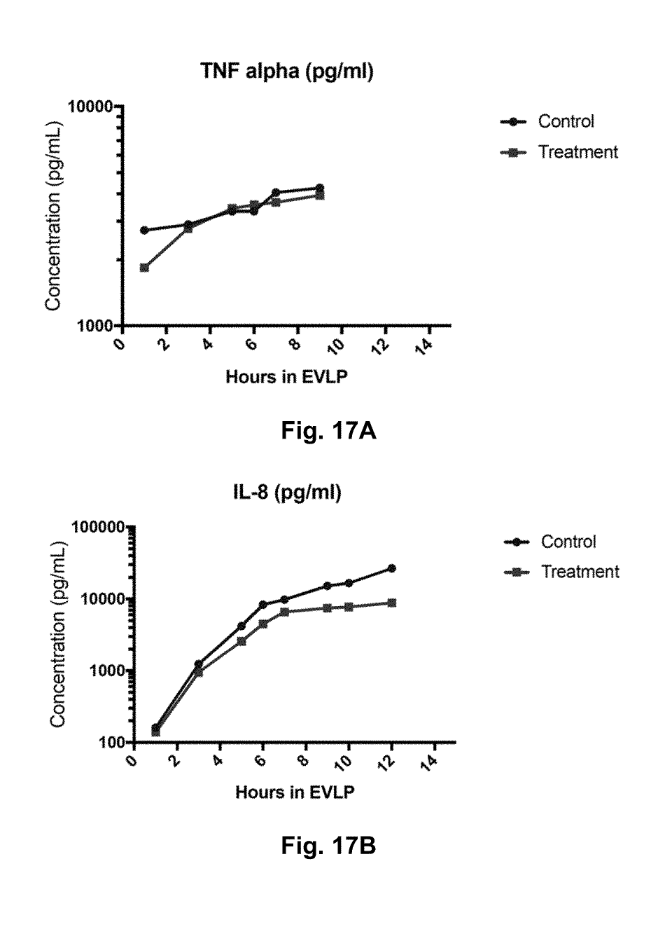

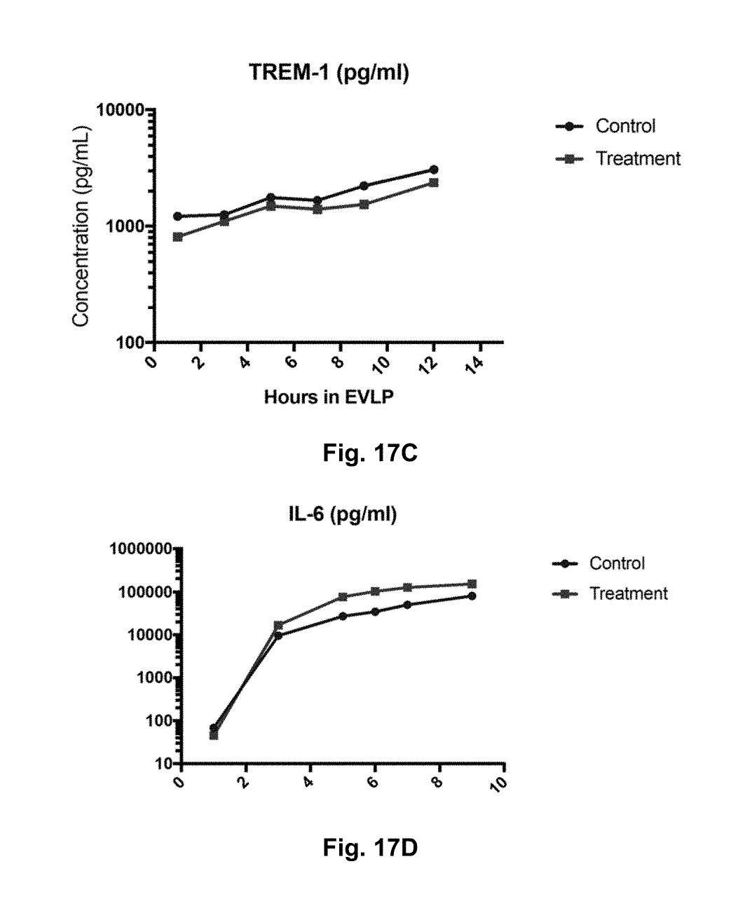

Case 1: Donor: 66 year old male who died after cardiac arrest. Cause of death: intracranial hemorrhage. Best pO₂: 354. The lungs were rejected for clinical transplantation because of aspiration concerns. The left lung showed recurrent purulent secretions during the donor surgery (S. aureus in culture) and signs of edema before EVLP. Donor chest X-ray showed consolidation of the left lower lobe. Left lung was randomly assigned to receive A1AT. Physiological lung function was evaluated hourly for 12 hours. PA pressure, LA pressure, peak airway pressure, plateau pressure, and dynamic and static compliance were recorded and analyzed. Perfusate gas analysis was performed in samples taken from the venous and arterial sides. The physiology of the right lung (untreated) was very stable during EVLP. The total Steen® loss during EVLP for the right lung was 1780 ml. The left lung (treated) physiology and oxygenation during the first 5 hours of EVLP were not good and declined every hour. After the 5^{th} hour the lung recovered improving the delta pO₂. Total Steen® loss during EVLP was 850 ml. (See FIG. 13.) The airway pressure was lower and compliance higher in the control group. (See FIGS. 14A and B.) We measured the A1AT content in perfusate and tissue and saw relatively stable levels during the 12 hours of EVLP. (See FIG. 15A-15C.) Caspase 3 decreased over time on the treated lung and increased on the untreated lung. At the end of EVLP (12 h) caspase 3 was lower on the treatment lung. (See FIG. 16A-16B.) Cytokines (IL-6, IL-8, TNF alpha and TREM-1) were measured in hourly perfusate. The treated lung showed lower levels of IL-8, TNF alpha and TREM-1 than the control lung. IL-6 was higher in the treated lung. (See FIG. 17A-17D.)

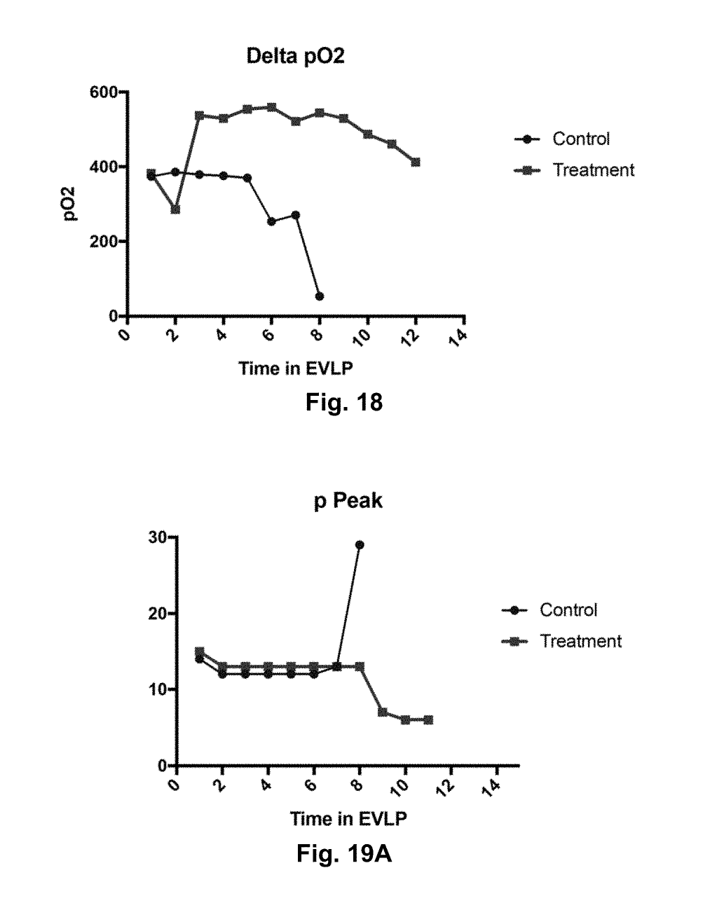

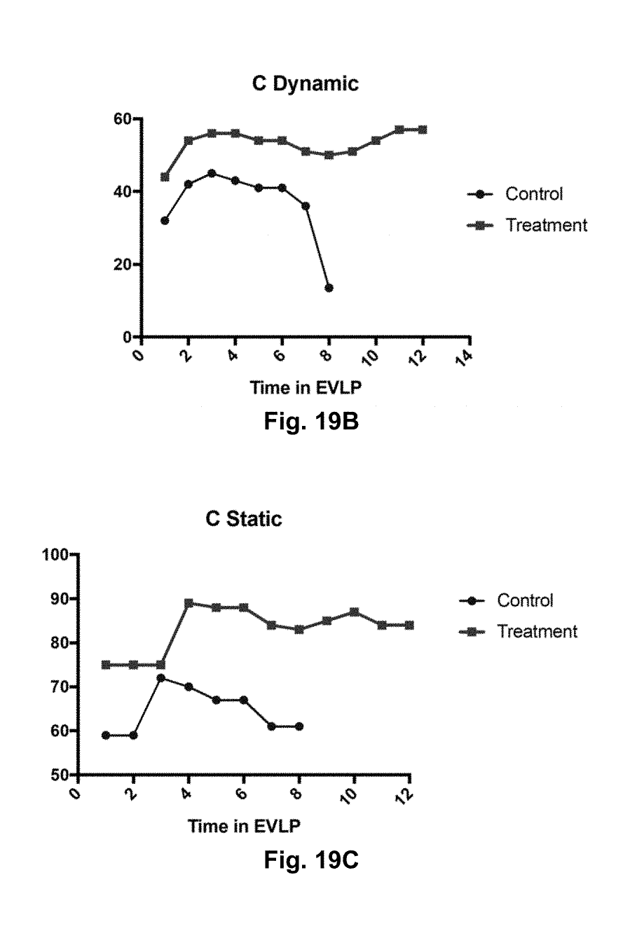

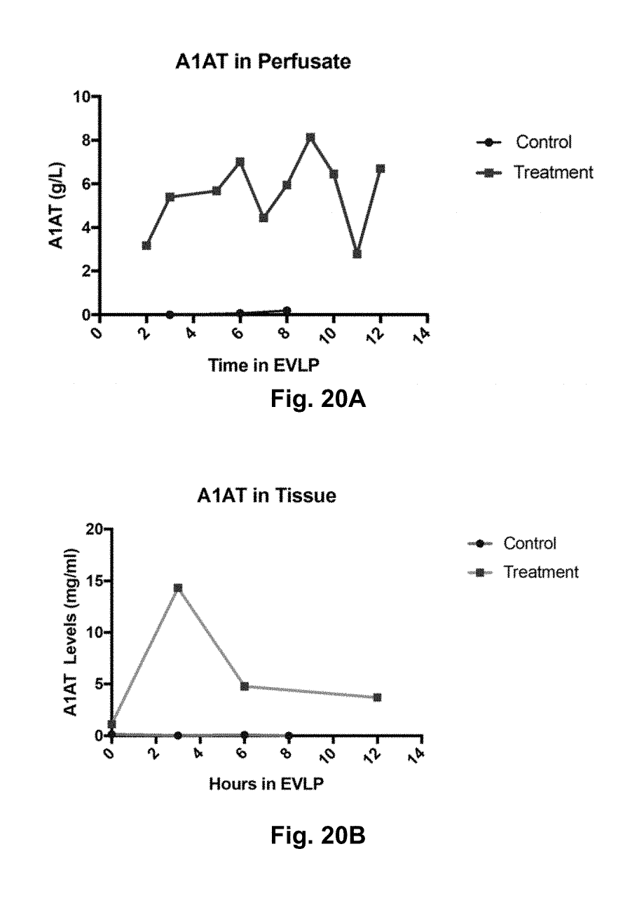

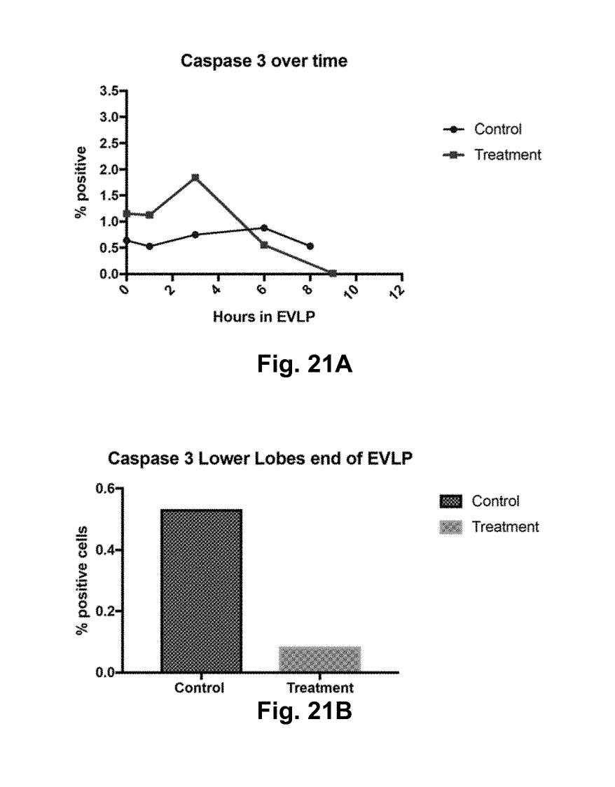

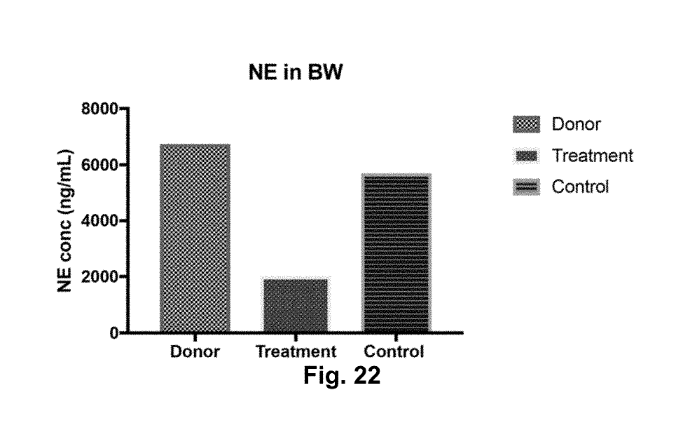

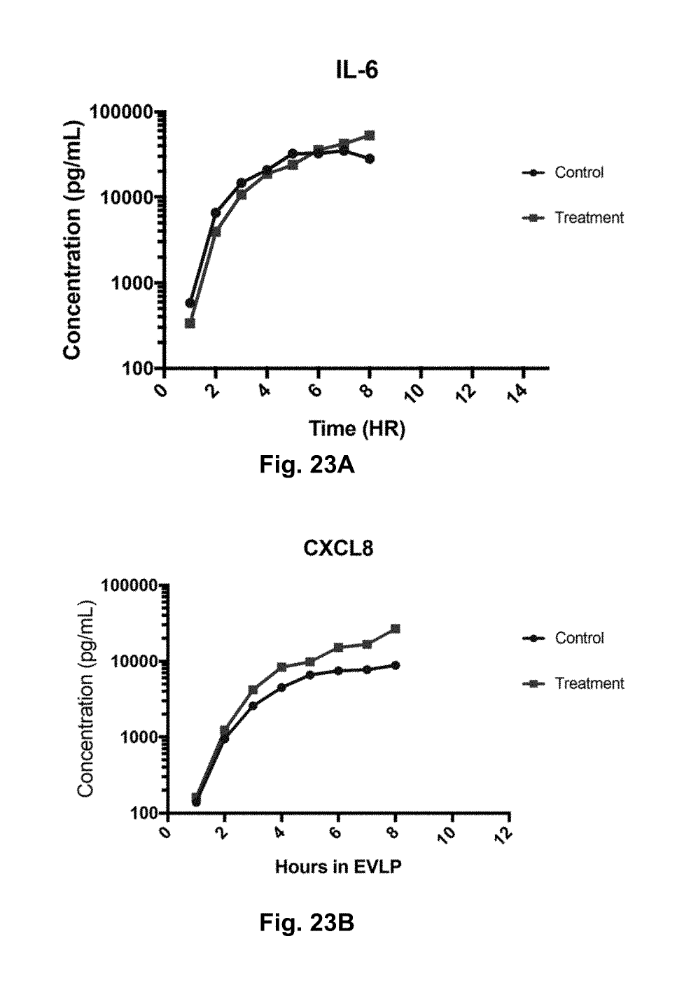

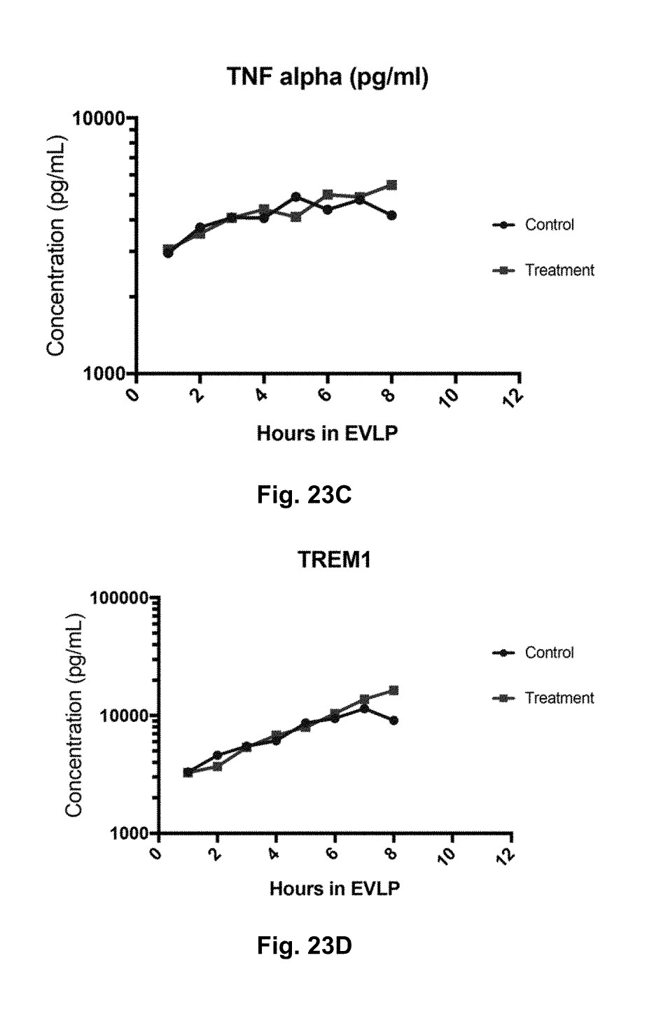

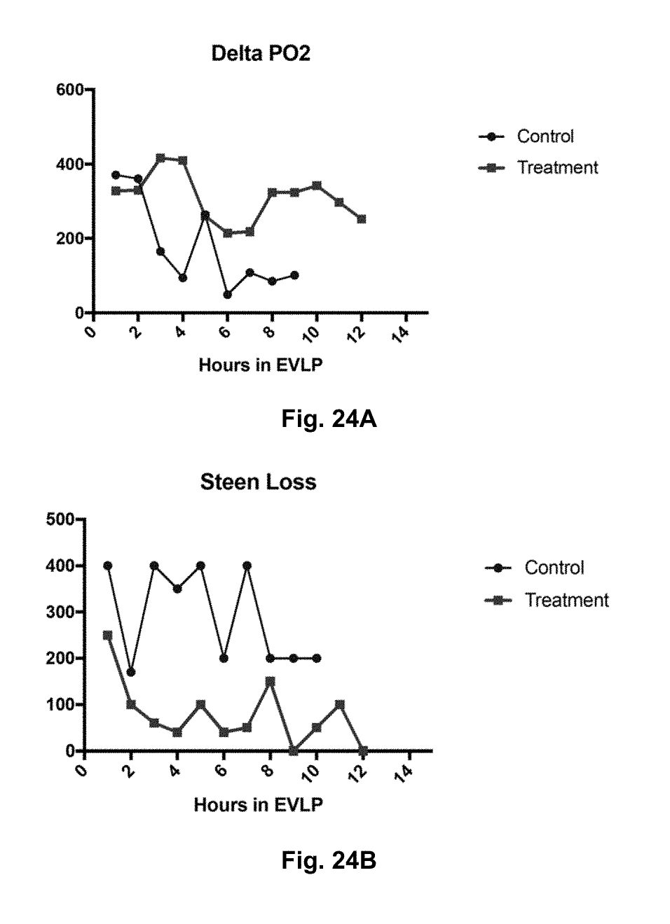

Case 2: Donor: 50 year old male. Brain death donor that arrested during donor surgery. Best pO₂: <200. The lungs were rejected for clinical transplantation because of low pO₂. Recurrent purulent secretions specially on the right side during the donor surgery. Right upper and lower lobes were heavy. Nodule on the right lower lobe sent to pathology showed pneumonia. Donor chest X-ray showed consolidation of the right lower lobe. Right lung was randomly assigned to receive A1AT. The physiology of the right lung (treated) improved during EVLP. The total Steen® loss during EVLP for the right lung was 300 ml. The left lung physiology and oxygenation during the first 5 hours of EVLP were stable and declined after the sixth hour peak pressure pO₂ decreased. The left lung consumed too much Steen®, and the EVLP for this lung was stopped. Total Steen® loss during EVLP for the left lung 3300 ml. FIG. 18 shows the delta pO₂ during EVLP for case 2 in the treatment and control lungs. The airway pressure was lower and compliance higher in the treated lung. (See FIG. 19A-19C.) The A1AT content in perfusate and tissue was measured achieving relatively stable levels during the 12 hours of EVLP for the treated lung. The A1AT levels in the untreated lung are too low to be read. (See FIG. 20A-20B.) After comparing the A1AT levels achieved in both cases (1 and 2) we decided to use the same dose for the next 3 cases. Caspase 3 started higher in the right lung (treated) and decreased over time and increased on the untreated lung. (See FIG. 21A-21B.) At the end of EVLP caspase 3 was lower on the treatment lung. For the second case we had donor bronchial wash as well as bronchial wash at the end of EVLP (right and left lungs). We performed a neutrophil elastase ELISA assay to measure the NE content and saw that the NE content decreased after treatment. (See FIG. 22.) Cytokines (IL-6, IL-8, TNF alpha and TREM-1) were measured in hourly perfusate, the cytokines levels seem to be higher in the treatment lung but the data shown here are not corrected for important dilution factors (Steen® was added just to the control lung). (See FIG. 23A-23D.)

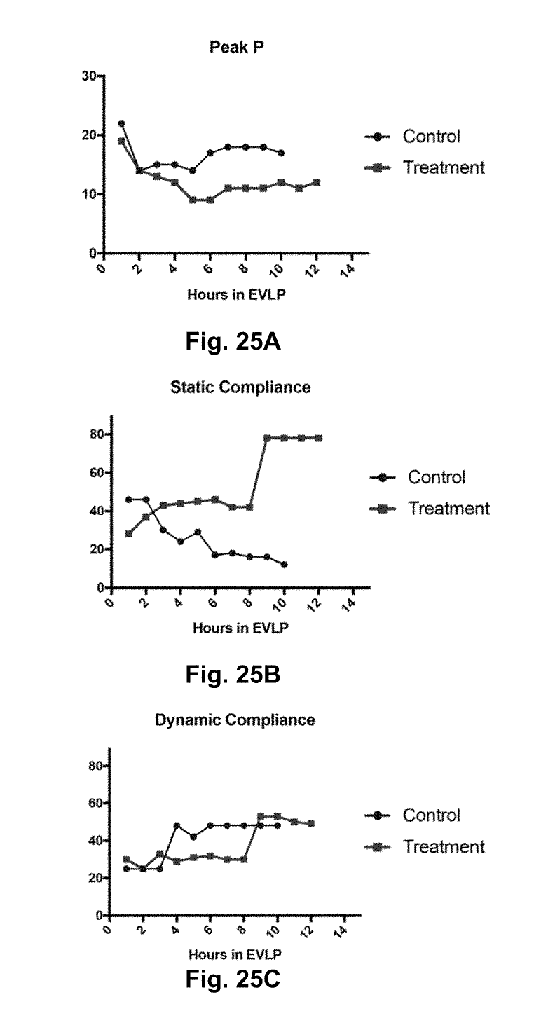

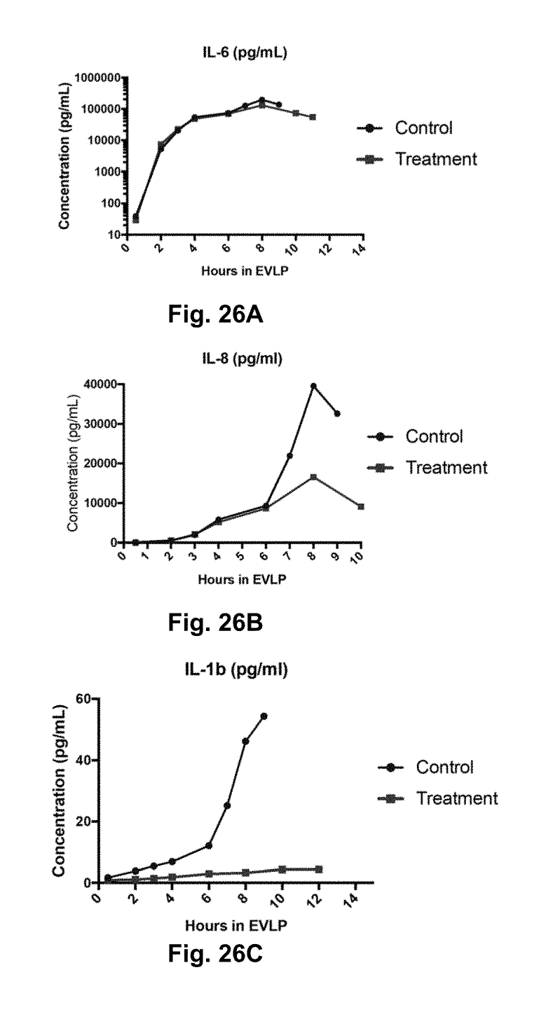

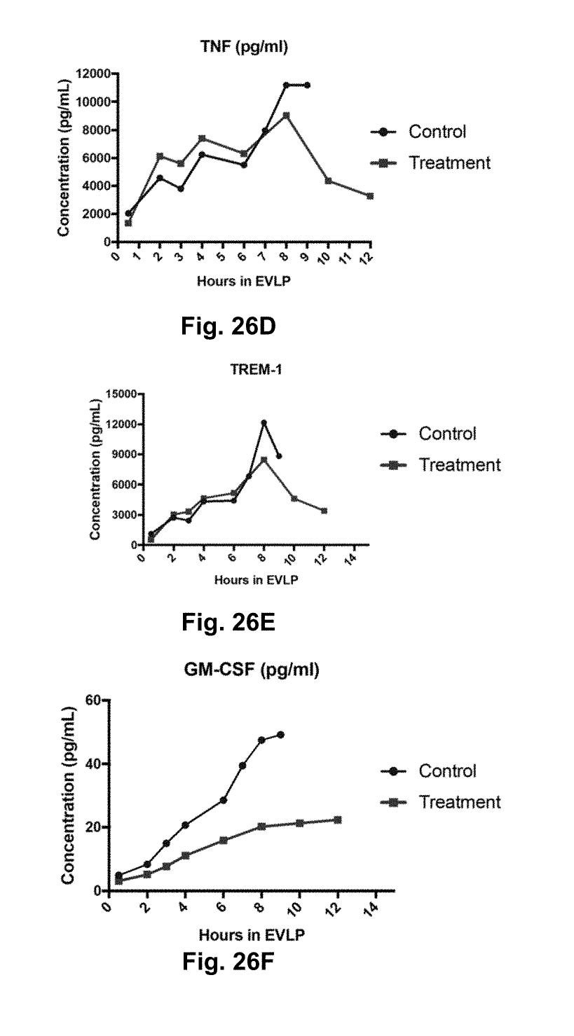

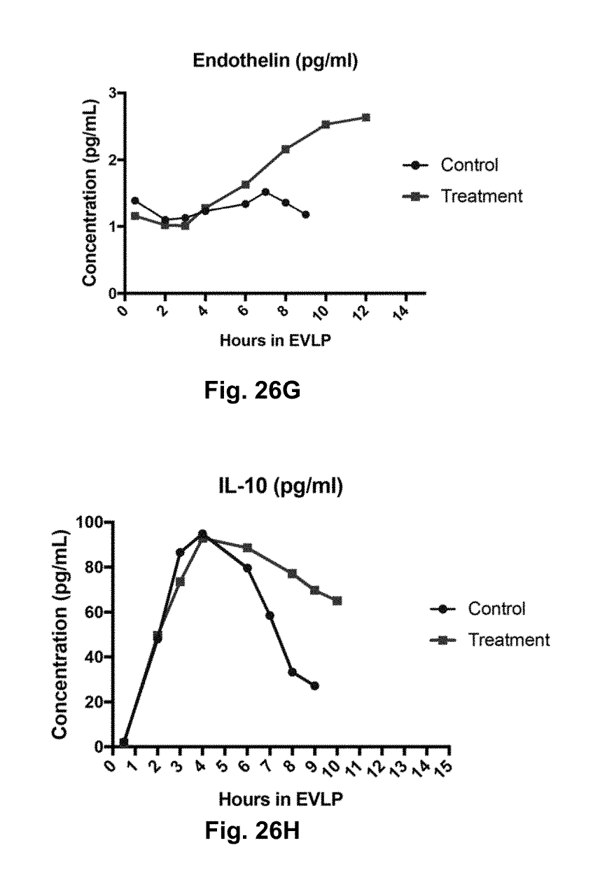

Case 3: Donor: 49 year old female. Brain death donor. pO₂ before donor surgery: 91. The lungs were rejected for clinical transplantation because of low pO₂ and heavy smoking history (1.5 packs/day for 28 years). Bronchoscopy: moderate purulent secretions and erythema both sides. Donor chest CT showed consolidation of the posterior segments of both lungs. Left lung was randomly assigned to receive A1AT. The physiology of the right lung (control) was better at the beginning, but got worse over time and the EVLP had to be stopped at 9 hours. The total Steen® loss during EVLP for the right lung was 2940 ml. The left lung physiology and oxygenation improved during the first 4 hours of EVLP declined at 4 to 6^{th} hour and improved back during the next 6 hours. The left lung consumed 1860 ml of Steen®. (See FIG. 24A-24B.) The airway pressure was lower and compliance higher in the treated lung as shown in FIG. 25A-25C. Cytokines (IL-6, IL-8, TNF alpha, TREM-1, IL-10, Endothelin, IL-1 beta, GM-CFS) were measured in hourly perfusate. (See FIG. 26A-26H.) Pro inflammatory cytokines: IL-6, IL-8, IL-1 beta, TNF alpha, TREM-1 and GM-CSF were lower in the treated lung, endothelin was higher in the treatment group. Anti-inflammatory cytokine IL-10 was higher in the treated lung. In this case Steen® solution was also added to the non treated lung and the actual levels of cytokines for the non treated lung will be probably higher once we correct for dilution. After EVLP the control lung showed complete consolidation of the lower lobe.

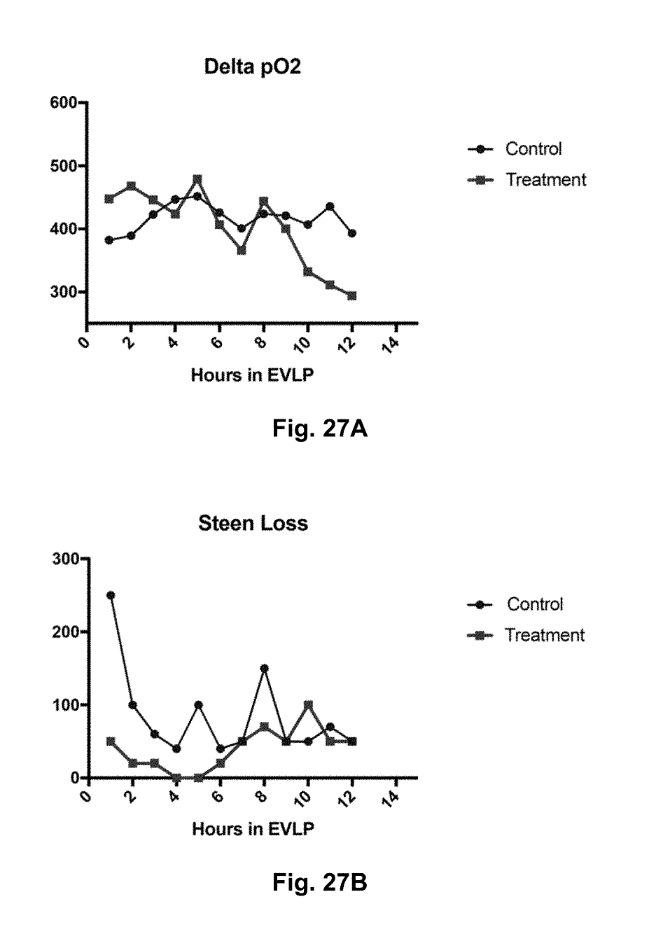

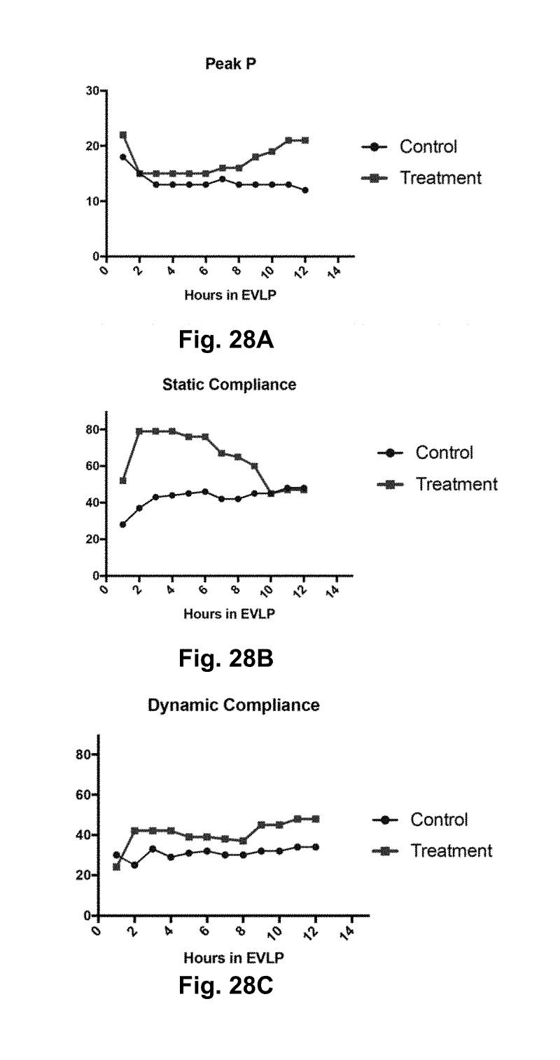

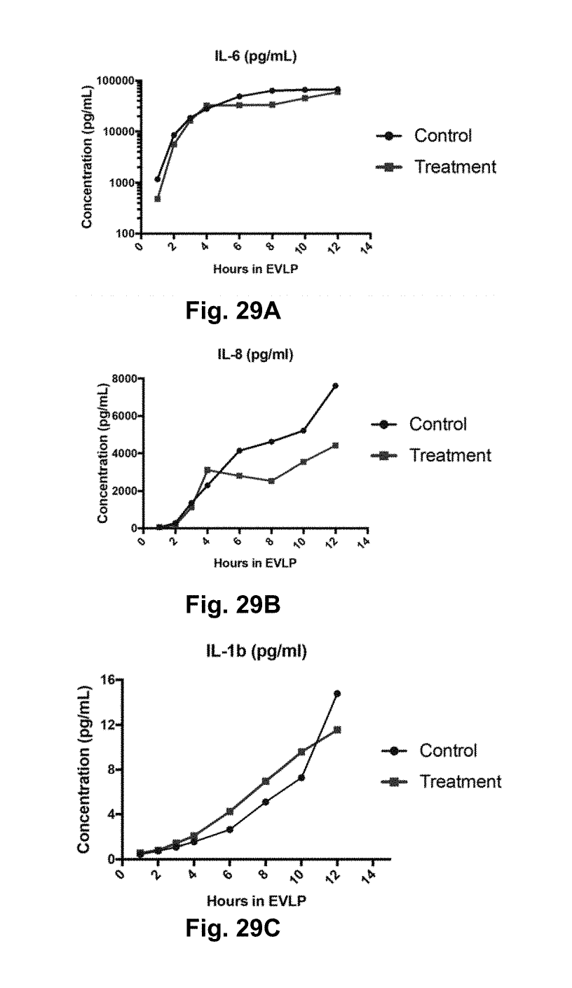

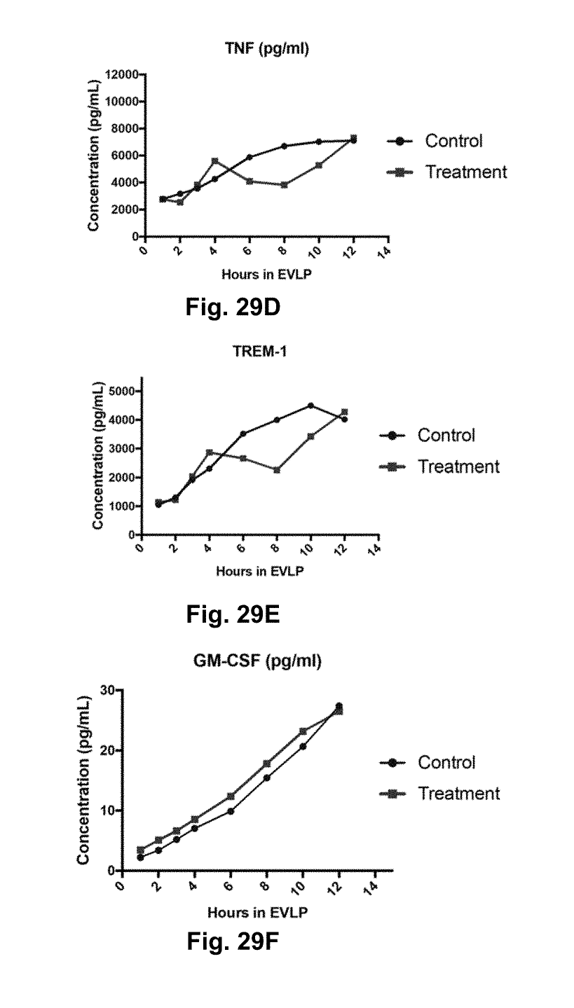

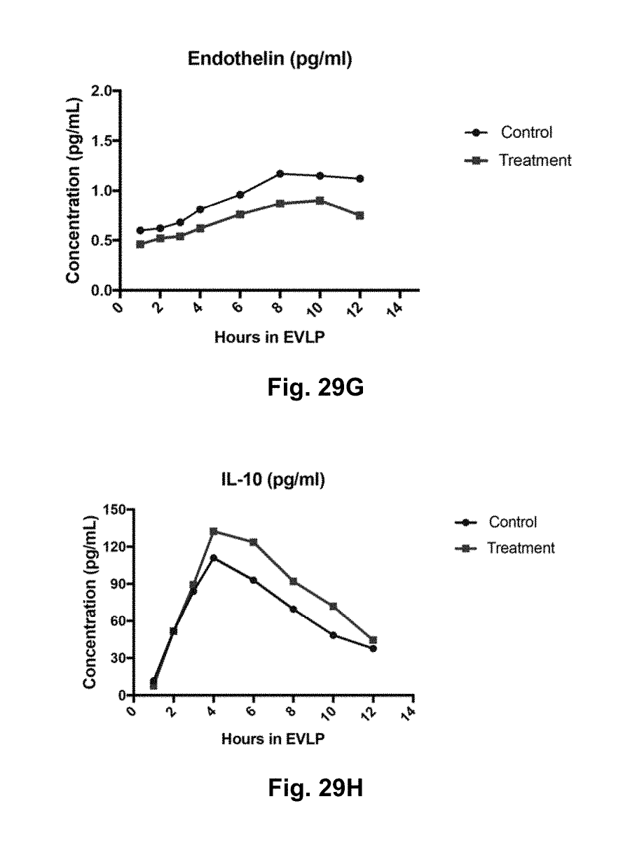

Case 4: Donor: 25 year old female. Brain death donor. Cause of death: anaphylactic reaction. pO₂ before donor surgery: 398. The lungs were rejected for clinical transplantation because of high airway pressure and low compliance. Bronchoscopy: mild to moderate purulent secretions and erythema both sides. Right lower lobe showed consolidation in chest x-ray and was evident at donor surgery. Right lung was assigned to receive A1AT. The delta pO₂ in the treated group decreased over time. The total Steen® loss during EVLP for the right lung (treatment) was 480 ml. The left lung physiology and oxygenation was stable during EVLP. The left lung lost 870 ml of Steen®. (FIG. 27A-27B.) Peak airway pressure started higher in the treated lung, decreased the second hour and was stable until the 8^{th} hour when it increased again. Static compliance started higher in the treated lung and decreased over time. Dynamic compliance started lower in the treated lung and ended up higher. (See FIG. 28A-28C.) Cytokines (IL-6, IL-8, TNF alpha, TREM-1, IL-10, Endothelin, IL-1 beta, GM-CFS) were measured in hourly perfusate. (See FIG. 29A-29H.) Pro inflammatory cytokines: IL-6, IL-8, IL-1 beta, TNF alpha, GM-CSF and endothelin were lower in the treated lung, TREM-1 was higher in the treated lung. Anti-inflammatory cytokine IL-10 was higher in the treated lung. In this case Steen® solution was also added to the non treated lung and the actual levels of cytokines for the non treated lung will be probably higher once corrected for dilution.

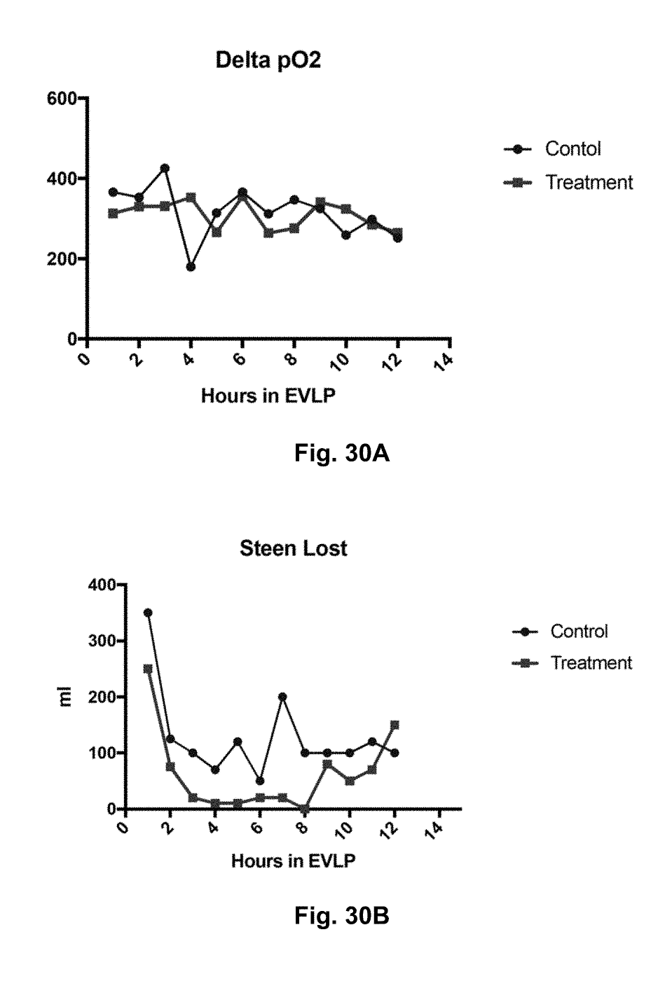

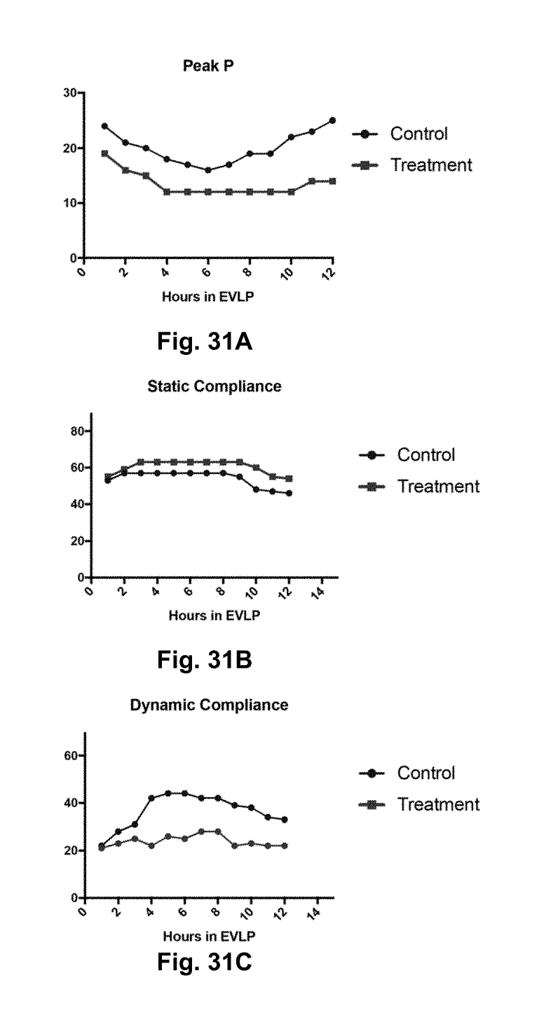

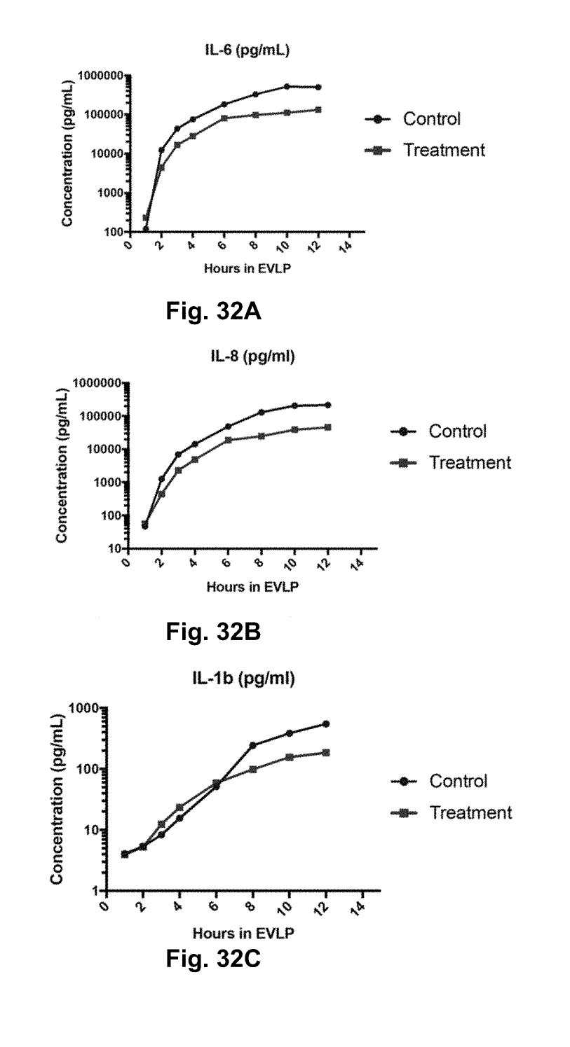

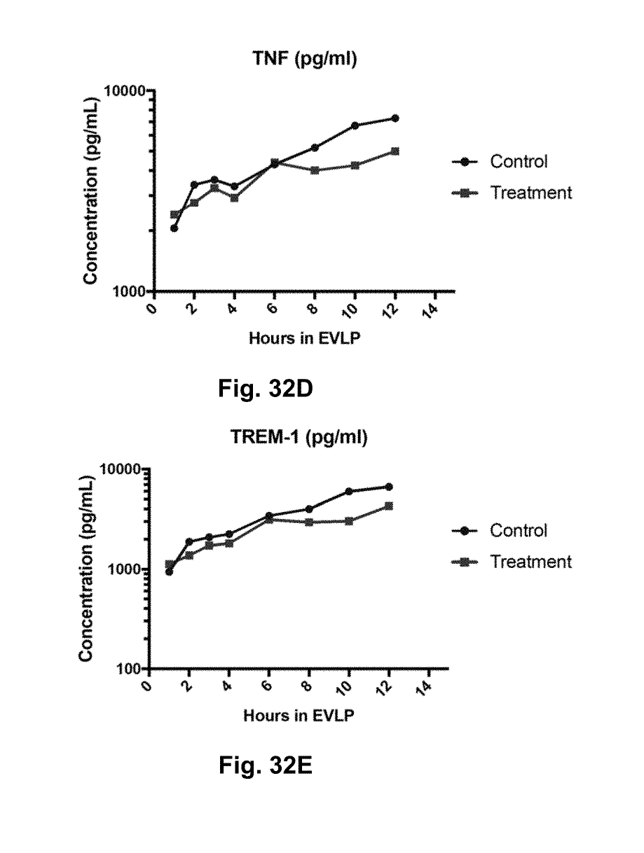

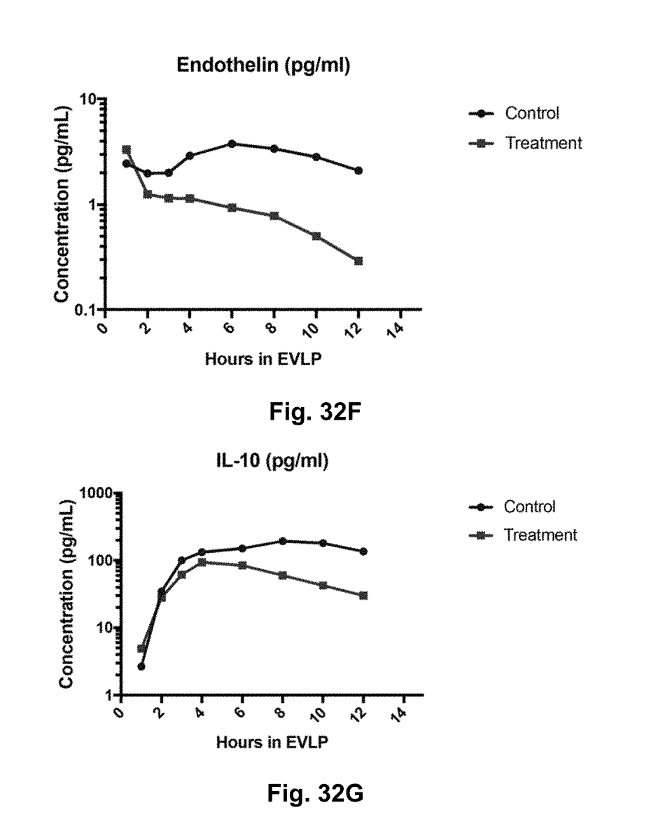

Case 5: Donor: 58 year old male. DCD donor. Cause of death: cardiac arrest. Best pO₂: 371. The lungs were rejected for clinical transplantation because of signs of emphysema. Bronchoscopy: heavy growth of S. pneumonia. Left lung was assigned to receive A1AT. The delta pO₂ in the treated group started lower and remained stable during EVLP. (FIG. 30A.) Peak airway pressure started and remained lower in the treated lung. Static compliance was higher in the treated lung. Dynamic compliance was lower in the treated lung. (See FIG. 31B-31C.) The total Steen® loss during EVLP for the right lung (treatment) was 755 ml. The left lung (control) lost a total of 1585 ml of Steen®. (FIG. 30B.) Cytokines (IL-6, IL-8, TNF alpha, TREM-1, IL-10, Endothelin, IL-1 beta, GM-CFS) were measured in hourly perfusate. Pro inflammatory cytokines: IL-6, IL-8, IL-1 beta, TNF alpha, TREM-1, and endothelin were lower in the treated lung. Anti-inflammatory cytokine IL-10 at the end of EVLP was lower in the control lung. (See FIG. 32A-32G.) In this case, more Steen® solution was also added to the non treated lung compared with the amount of Steen® added to the circuit of the treated lung the actual levels of cytokines for the non treated lung will be probably higher once corrected for dilution.

Data from our initial study demonstrated that A1AT potentially decreases the inflammatory response, the IRI, and the apoptosis signaling. The aim of this study was to further investigate the effect of A1AT as an anti-inflammatory and anti-apoptotic agent using EVLP as a delivery platform in a more severely injured human lung, and subsequently translate it to a clinical trial if the results are positive. Using human lungs has advantages, since treating human lungs with human A1AT removes any effects due to species differences. These experiments test the impact of perfusion with A1AT in lungs that suffered the process of brain death, which may lead to inflammation, as well as lungs that went through the cardiac death process. The lungs tested in these experiments were also of poor quality, having been rejected for clinical transplantation. Even though each set of treatment and control lungs assayed in the 5 above cases comes from the same donor and suffered some of the insults in the same manner, the injuries that each lung presents in comparison to the other lung from the same donor can be quite different, making comparisons between two lungs from the same donor difficult.

After the first 2 cases we confirmed that the dose of A1AT selected using earlier pig studies is sufficient to maintain relatively stable levels of A1AT both in perfusate and in lung tissue. Even though each case that we have used to test the drug was rejected for transplantation for very different reasons and each set of donor lungs suffered from very different insults, addition of A1AT to the perfusate showed potential benefits in terms of lung physiology during EVLP as well as decrease of pro-inflammatory cytokines tested in perfusate. (See the table below.) Pro and anti-inflammatory cytokines in lung tissue, bronchial wash and EVLP perfusate will now be tested using PCR and ELISA. All the lungs treated with A1AT showed less Steen loss than the untreated control lungs, which could be the result of a protective effect of A1AT over the endothelium.

A summary of the data from the above 5 cases is shown in the table below.

| | | | | Reason for | | A1AT- | | |
|---|---|---|---|---|---|---|---|---|
| | | | | being | | Treated | | |
| | | | | Declined (for | | Lung | | |
| | | Donor | | EVLP and/or | Other Relevant | (LL or | | |
| Case | Donor COD | Type | TLC | Tx) | Donor Notes | RL) | EVLP Notes | Cytokines |
| #1 | Intracranial | DCD | 7.76 | Broncho | Left lung with | Left | Left lung almost crashed at 4 h. | Treatment had lower TNF |
| | hemorrhage | | | aspiration | purulent | | Recovered after 5 h | alpha, IL-8 and TREM-1. |
| | | | | | secretions and | | | Higher IL-6 |
| | | | | | edema. | | | |
| | | | | | Consolidation in | | | |
| | | | | | Chest x-ray | | | |
| #2 | Intracranial | NDD | 7.04 | Low pO₂ | Purulent secretion | Right | Right lung improved during EVLP. | Treatment had higher IL-6, |
| | hemorrhage | | | | and consolidation | | Left lung “crashed” at 8 h. Left lung | IL-8, TNF-alpha and TREM-1 |
| | | | | | right side. | | consumed a lot of Steen | |
| | | | | | Smoker | | | |
| | | | | | 15 half pack/15 | | | |
| | | | | | years | | | |
| #3 | Head and | NDD | 6.54 | Heavy | Bilateral | Left | Right lung started better and got | Treatment had Lower IL-6, |
| | chest | | | smoker + | contusion and | | worse over time EVLP had to be | IL-8, IL-1 beta, TNF alpha |
| | trauma | | | low pO₂ | pneumothorax | | stopped at 9 h. Control lost more | TREM-1 GM-CSF and |
| | | | | | | | steen | Endothelin and higher IL-10 |
| #4 | Anaphylactic | NDD | 5.92 | High airway | Right lung mild | Right | Peak airway pressure started | Treatment had lower IL-6, |
| | reaction | | | pressure + | basal | | higher in the treated lung. | IL-8, IL-1 beta, TNF alpha, |
| | | | | low | consolidation | | Dynamic compliance started lower | GM-CSF and Endothelin. |
| | | | | compliance | | | in the treated lung and ended up | Higher TREM-1 and higher |
| | | | | | | | higher. Control lost more Steen. | IL-10 |
| #5 | Cardiac | DCD | 7.13 | Emphysema | Heavy grow on S | Left | Delta pO₂ in the treated group | Treatment had lower IL-6, |
| | arrest | | | | pneumoniae in | | started lower and remained | IL-8 IL-1 beta, TNF-alpha, |
| | | | | | bronchial wash. | | stable. Peak pressure started and | TREM-1, Endothelin and IL- |
| | | | | | Heavy smoker | | remained lower in the treated | 10 |
| | | | | | | | lung. Static compliance was | |
| | | | | | | | higher in the treated lung. | |
| | | | | | | | Dynamic compliance was lower in | |
| | | | | | | | the treated lung. More Steen | |
| | | | | | | | added to control | |

### REFERENCES

1. Punch, J. D., et al., Organ donation and utilization in the United States, 1996-2005. Am J Transplant, 2007. 7(5 Pt 2): p. 1327-38.
2. Cypel, M., et al., Technique for prolonged normothermic ex vivo lung perfusion. J Heart Lung Transplant, 2008. 27(12): p. 1319-25.
3. Cypel, M. and S. Keshavjee, Extending the donor pool: rehabilitation of poor organs. Thorac Surg Clin, 2015. 25(1): p. 27-33.
4. Lee, J. W., et al., Allogeneic human mesenchymal stem cells for treatment of E. coli endotoxin-induced acute lung injury in the ex vivo perfused human lung. Proc Natl Acad Sci USA, 2009. 106(38): p. 16357-62.
5. Mordant, P., et al., Mesenchymal stem cell treatment is associated with decreased perfusate concentration of interleukin-8 during ex vivo perfusion of donor lungs after 18-hour preservation. J Heart Lung Transplant, 2016. 35(10): p. 1245-1254.
6. Cypel, M., et al., Functional repair of human donor lungs by IL-10 gene therapy. Sci Trans! Med, 2009. 1(4): p. 4ra9.
7. Machuca, T. N., et al., Safety and Efficacy of Ex Vivo Donor Lung Adenoviral IL-10 Gene Therapy in a Large Animal Lung Transplant Survival Model. Hum Gene Ther, 2017, doi: 10.1089/hum.2016.070.
8. Yeung, J. C., et al., Ex vivo adenoviral vector gene delivery results in decreased vector-associated inflammation pre- and post-lung transplantation in the pig. Mol Ther, 2012. 20(6): p. 1204-11.
9. Nakajima, D., et al., Ex Vivo Perfusion Treatment of Infection in Human Donor Lungs. Am J Transplant, 2016. 16(4): p. 1229-37.
10. Andreasson, A., et al., The effect of ex vivo lung perfusion on microbial load in human donor lungs. J Heart Lung Transplant, 2014. 33(9): p. 910-6.11.
Nakajima, D., et al., Lung Lavage and Surfactant Replacement During Ex Vivo Lung Perfusion for Treatment of Gastric Acid Aspiration Induced Donor Lung Injury. J Heart Lung Transplant, 2016. 36(5): p. 577-585.
12. Inci, I., et al., Reconditioning of an injured lung graft with intrabronchial surfactant instillation in an ex vivo lung perfusion system followed by transplantation. J Surg Res, 2013. 184(2): p. 1143-9.
13. Machuca, T. N., et al., Injury-specific ex vivo treatment of the donor lung: pulmonary thrombolysis followed by successful lung transplantation. Am J Respir Crit Care Med, 2013. 188(7): p. 878-80.
14. Inci, I., et al., Successful lung transplantation after donor lung reconditioning with urokinase in ex vivo lung perfusion system. Ann Thorac Surg, 2014, 98(5): p. 1837-8.
15. Tang, P. S., et al., Acute lung injury and cell death: how many ways can cells die? Am J Physiol Lung Cell Mol Physiol, 2008. 294(4): p. L632-41.
16. Okutani, D., et al., Src protein tyrosine kinase family and acute inflammatory responses. Am J Physiol Lung Cell Mol Physiol, 2006. 291(2): p. L129-41.
17. Kim, H., et al., δV1-1 Reduces Pulmonary Ischemia Reperfusion-Induced Lung Injury by Inhibiting Necrosis and Mitochondrialk Localization of PKCδ and p53. Am J Transplant, 2016. 16(1): p. 83-98.
18. Andrade, C. F., et al., Innate immunity and organ transplantation: the potential role of toll-like receptors. Am J Transplant, 2005. 5(5): p. 969-75.
19. Gettins, P. G., Serpin structure, mechanism, and function. Chem Rev, 2002. 102(12): p. 4751-804.
20. Campos, M. A., et al., Effects of a disease management program in individuals with alpha-1 antitrypsin deficiency. COPD, 2009. 6(1): p. 31-40.
21. Iskender, I., et al., Human alpha(1)-antitrypsin improves early post-transplant lung function: Pre-clinical studies in a pig lung transplant model. Journal of Heart and Lung Transplantation, 2016. 35(7): p. 913-921.
22. Gao, W., et al., alpha1-Antitrypsin inhibits ischemia reperfusion-induced lung injury by reducing inflammatory response and cell death. J Heart Lung Transplant, 2014. 33(3): p. 309-15.
23. Nita, I. M., D. Serapinas, and S. M. Janciauskiene, alpha1-Antitrypsin regulates CD14 expression and soluble CD14 levels in human monocytes in vitro. Int J Biochem Cell Biol, 2007. 39(6): p. 1165-76.
24. Churg, A., et al., Alpha-1-antitrypsin and a broad spectrum metalloprotease inhibitor, RS113456, have similar acute anti-inflammatory effects. Lab Invest, 2001. 81(8): p. 1119-31.
25. Bergin, D. A., et al., alpha-1 Antitrypsin regulates human neutrophil chemotaxis induced by soluble immune complexes and IL-8. J Clin Invest, 2010. 120(12): p. 4236-50.
26. Lewis, E. C., et al., alpha1-Antitrypsin monotherapy induces immune tolerance during islet allograft transplantation in mice. Proc Natl Acad Sci USA, 2008. 105(42): p. 16236-41.
27. Toldo, S., et al., Alpha-1 antitrypsin inhibits caspase-1 and protects from acute myocardial ischemia-reperfusion injury. J Mol Cell Cardiol, 2011. 51(2): p. 244-51.
28. Chen, G., et al., ECDI-fixed allogeneic splenocytes combined with alpha1-antitrypsin prolong survival of rat renal allografts. Int Immunopharmacol, 2015. 26(1): p. 43-9.
29. Zager, R. A., A. C. Johnson, and K. B. Frostad, Rapid renal alpha-1 antitrypsin gene induction in experimental and clinical acute kidney injury. PLoS One, 2014. 9(5): p. e98380.
30. Jedicke, N., et al., alpha-1-antitrypsin inhibits acute liver failure in mice. Hepatology, 2014. 59(6): p. 2299-308.
31. Machuca, T. N. and M. Cypel, Ex vivo lung perfusion. J Thorac Dis, 2014. 6(8): p. 1054-62.
32. Klinzing, S., et al., Wet-to-dry ratio of lung tissue and surfactant outwash after one-lung flooding. Res Exp Med (Berl), 2000. 200(1): p. 27-33.
33. Cypel, M., et al., Normothermic ex vivo perfusion prevents lung injury compared to extended cold preservation for transplantation. Am J Transplant, 2009. 9(10): p. 2262-9.
34. Nishina, K., et al., ONO-5046, an elastase inhibitor, attenuates endotoxin-induced acute lung injury in rabbits. Anesth Analg, 1997. 84(5): p. 1097-103.
35. Wijsman, J. H., et al., A new method to detect apoptosis in paraffin sections: in situ end-labeling of fragmented DNA. J Histochem Cytochem, 1993. 41(1): p. 7-12.
36. Machuca, T. N., et al., Protein expression profiling predicts graft performance in clinical ex vivo lung perfusion. Ann Surg, 2015. 261(3): p. 591-7.
37. Gao X, L. W., and Liu M., Ex Vivo Lung Perfusion: Scientific Research and Clinical Application. Practical Journal of Organ Transplantation, 2017, 5(3): p. 177-178.
38. Gennai, S., et al., Effects of cyclosporine a in ex vivo reperfused pig lungs. Microcirculation, 2014. 21(1): p. 84-92.
39. Valenza, F., et al., beta-adrenergic agonist infusion during extracorporeal lung perfusion: effects on glucose concentration in the perfusion fluid and on lung function. J Heart Lung Transplant, 2012. 31(5): p. 524-30.
40. Emaminia, A., et al., Adenosine A(2)A agonist improves lung function during ex vivo lung perfusion. Ann Thorac Surg, 2011. 92(5): p. 1840-6.
41. Nakajima, D., et al., Lung Lavage and Surfactant Replacement During Ex Vivo Lung Perfusion for Treatment of Gastric Acid Aspiration Induced Donor Lung Injury. J Heart Lung Transplant, 2017. 36(5): p. 577-585.
42. Haam, S., et al., The effects of hydrogen gas inhalation during ex vivo lung perfusion on donor lungs obtained after cardiac death. Eur J Cardiothorac Surg, 2015. 48(4): p. 542-7.
43. Kondo, T., et al., beta2-Adrenoreceptor Agonist Inhalation During Ex Vivo Lung Perfusion Attenuates Lung Injury. Ann Thorac Surg, 2015. 100(2): p. 480-6.
44. Harada, M., et al., A neutrophil elastase inhibitor improves lung function during ex vivo lung perfusion. Gen Thorac Cardiovasc Surg, 2015. 63(12): p. 645-51.
45. Charles, E. J., et al., Lungs donated after circulatory death and prolonged warm ischemia are transplanted successfully after enhanced ex vivo lung perfusion using adenosine A2B receptor antagonism. J Thorac Cardiovasc Surg, 2017 Apr. 12 pii: S0022-5223(17)30678-5. doi: 10.1016/j.jtcvs.2017.02.072.
46. Martens, A., et al., Immunoregulatory effects of multipotent adult progenitor cells in a porcine ex vivo lung perfusion model. Stem Cell Res Ther, 2017. 8(1): p. 159.
47. Lewis, E. C., et al., Alpha1-antitrypsin monotherapy prolongs islet allograft survival in mice. Proc Natl Acad Sci USA, 2005. 102(34): p. 12153-8.
48. Tawara, I., et al., Alpha-1-antitrypsin monotherapy reduces graft-versus-host disease after experimental allogeneic bone marrow transplantation. Proc Natl Acad Sci USA, 2012. 109(2): p. 564-9.
49. Soy D, de la Roza C, Lara B, Esquinas C, Tones A, Miravitlles M. Alpha-1-antitrypsin deficiency: optimal therapeutic regimen based on population pharmacokinetics. Thorax. 2006; 61(12):1059-64.
50. Iskender I, Sakamoto J, Nakajima D, Lin H, Chen M, Kim H, et al. Human alpha1-antitrypsin improves early post-transplant lung function: Pre-clinical studies in a pig lung transplant model. J Heart Lung Transplant. 2016; 35(7):913-21.
51. Yeung J C, Keshavjee S. Overview of clinical lung transplantation. Cold Spring Harb Perspect Med. 2014; 4(1):a015628.
52. Lopau K, Mark J, Schramm L, Heidbreder E, Wanner C. Hormonal changes in brain death and immune activation in the donor. Transpl Int. 2000; 13 Suppl 1:S282-5.
53. Mariscal A, Caldarone L, Tikkanen J, Nakajima D, Chen M, Yeung J, et al. Pig lung transplant survival model. Nat Protoc. 2018; 13(8):1814-28.
54. Gao W, Zhao J, Kim H, Xu S, Chen M, Bai X, et al. alpha1-Antitrypsin inhibits ischemia reperfusion-induced lung injury by reducing inflammatory response and cell death. J Heart Lung Transplant. 2014; 33(3):309-15.

1. A method of ex vivo organ perfusion, comprising perfusing an organ ex vivo with a perfusion solution comprising alpha-1-antitrypsin (A1AT), wherein the perfusion solution is exposed to an environment having a normothermic temperature.

2. A method of transplanting an organ, comprising:
a) perfusing an organ ex vivo with a perfusion solution comprising A1AT, wherein the perfusion solution is exposed to an environment having a normothermic temperature; and
b) transplanting the ex vivo perfused organ into a recipient.

3. (canceled)

4. (canceled)

5. (canceled)

6. (canceled)

7. (canceled)

8. The method of claim 1, wherein the organ is a lung, a heart, a liver, a kidney, a pancreas, or a small bowel.

9. The method of claim 8 claims, wherein the organ is a lung.

10. The method of claim 9, wherein the lung is ventilated.

11. (canceled)

12. (canceled)

13. The method of claim 1, wherein the perfusion solution is exposed to an environment having a normothermic temperature of from 30° C. to 38° C., of from 32° C. to 38° C., of from 34° C. to 38° C., of from 36° C. to 38° C., of 30° C., of 31° C., of 32° C., of 33° C., of 34° C., of 35° C., of 36° C., of 37° C., or of 38° C.

14. (canceled)

15. (canceled)

16. (canceled)

17. The method of claim 1, wherein the organ has been donated after brain death (DBD) or after cardiac death (DCD).

18. The method of claim 1, wherein before perfusing, the organ is processed, preserved, or stored in a below room temperature environment.

19. (canceled)

20. (canceled)

21. (canceled)

22. (canceled)

23. (canceled)

24. The method of claim 1, wherein the A1AT comprises human A1AT.

25. The method of claim 24, wherein the A1AT is obtained from pooled human plasma, is recombinant, or is a fusion molecule comprising A1AT and a fusion partner, wherein the fusion partner optionally comprises an Fc variant.

26. The method of claim 1, wherein the concentration of A1AT in the perfusion solution is from 0.5 mg/mL to 5 mg/mL, from 0.5 mg/mL to 10 mg/mL, from 1 mg/mL to 5 mg/mL, from 3 mg/mL to 10 mg/mL, from 5 mg/mL to 10 mg/mL, from 5 mg/mL to 15 mg/m, from 8 mg/mL to 12 mg/mL, from 10 mg/mL to 20 mg/mL, from 10 mg/mL to 15 mg/mL, or from 15 mg/mL to 20 mg/mL.

27. (canceled)

28. The method of claim 1, wherein the perfusion solution further comprises one or more of albumin, a dextran compound, or a combination of salts.

29. (canceled)

30. (canceled)

31. (canceled)

32. (canceled)

33. (canceled)

34. (canceled)

35. (canceled)

36. (canceled)

37. The method of claim 28, wherein the combination of salts comprises one or more of sodium ion, potassium ion, calcium ion, magnesium ion, hydrogen carbonate ion, chloride ion, sodium chloride, potassium chloride, calcium chloride, magnesium chloride, sodium dihydrogen phosphate, sodium bicarbonate, or sodium hydroxide.

38. The method of claim 28, wherein the concentration of each salt corresponds to its normal serum concentration in human or animal blood.

39. (canceled)

40. (canceled)

41. (canceled)

42. The method of claim 1, wherein physiological function of the organ at the termination of the ex vivo perfusion or at a time after the ex vivo perfusion is increased compared to the physiological function at a time before or during the ex vivo perfusion.

43. (canceled)

44. The method of claim 1, wherein the organ is a lung and wherein the pulmonary arterial pressure (PAP) at the termination of the ex vivo perfusion or at a time after the ex vivo perfusion is decreased compared to the PAP at a time before or during the ex vivo perfusion.

45. The method of claim 1, wherein the organ is a lung and wherein the pulmonary vascular resistance (PVR) at the termination of the ex vivo perfusion or at a time after the ex vivo perfusion is decreased compared to the PVR at a time before or during the ex vivo perfusion.

46. The method of claim 1, wherein the organ is a lung and wherein the peak inspiratory pressure (Ppeak) sampled at the termination of the ex vivo perfusion or at a time after the ex vivo perfusion is decreased compared to the Ppeak sampled at a time before or during the ex vivo perfusion.

47. The method of claim 1, wherein the organ is a lung and wherein the plateau airway pressure (Pplat) sampled at the termination of the ex vivo perfusion or at a time after the ex vivo perfusion is decreased compared to the Pplat sampled at a time before or during the ex vivo perfusion.

48. The method of claim 1, wherein the organ is a lung and wherein the dynamic pulmonary compliance (Cdyn) sampled at the termination of the ex vivo perfusion or at a time after the ex vivo perfusion is (a) increased compared to the Cdyn sampled at a time before or during the ex vivo perfusion, or (b) maintained compared to the Cdyn sampled at a time before or during the ex vivo perfusion.

49. The method of claim 1, wherein the organ is a lung and wherein the static pulmonary compliance (Cstat) sampled at the termination of the ex vivo perfusion or at a time after the ex vivo perfusion is (a) increased compared to the Cstat sampled at a time before or during the ex vivo perfusion, or (b) maintained compared to the Cstat sampled at a time before or during the ex vivo perfusion.

50. The method of claim 1, wherein the organ is a lung and wherein the ratio of arterial oxygen partial pressure to fractional inspired oxygen (PaO2/FiO2) at the termination of the ex vivo perfusion or at a time after the ex vivo perfusion is (a) increased compared to the PaO2/FiO2 at a time before or during the ex vivo perfusion, or (b) maintained compared to the PaO2/FiO2 at a time before or during the ex vivo perfusion.

51. The method of claim 1, wherein the organ is a lung and wherein the ratio of arterial oxygen partial pressure to fractional inspired oxygen (PaO2/FiO2) before the ex vivo perfusion is less than 250 mmHg, less than 275 mmHg, less than 300 mmHg, less than 325 mmHg, less than 350 mmHg, or less than 400 mmHg.

52. The method of claim 1, wherein the organ is a lung and wherein the ratio of arterial oxygen partial pressure to fractional inspired oxygen (PaO₂/FiO₂) during the ex vivo perfusion, at the termination of the ex vivo perfusion, or at a time after the ex vivo perfusion is at least 300 mmHg or greater, at least 325 mmHg or greater, at least 350 mmHg or greater, at least 375 mmHg or greater, at least 400 mmHg or greater, at least 425 mmHg or greater, at least 450 mmHg or greater, at least 475 mmHg or greater, or at least 500 mmHg or greater.

53. (canceled)

54. The method of claim 1, wherein the organ is a lung and wherein the difference in oxygen pressure (PO2) between the left atrium (LA) and pulmonary artery (PA) (Delta PO2) at the termination of the ex vivo perfusion or at a time after the ex vivo perfusion is (a) increased compared to the Delta PO2 at a time before or during the ex vivo perfusion, or (b) maintained compared to the Delta PO2 at a time before or during the ex vivo perfusion.

55. A perfusion solution comprising (a) albumin, (b) a dextran compound, (c) a combination of salts, and (d) alpha-1-antitrypsin (A1AT).

56. The perfusion solution of claim 55, wherein the A1AT comprises human A1AT.

57. The perfusion solution of claim 56, wherein the A1AT is obtained from pooled human plasma, is recombinant, or is a fusion molecule comprising A1AT and a fusion partner, wherein the fusion partner optionally comprises an Fc variant.

58. The perfusion solution of claim 55, wherein the concentration of A1AT is from 0.5 mg/mL to 5 mg/mL, from 0.5 mg/mL to 10 mg/mL, from 1 mg/mL to 5 mg/mL, from 3 mg/mL to 10 mg/mL, from 5 mg/mL to 10 mg/mL, from 5 mg/mL to 15 mg/mL, from 8 mg/mL to 12 mg/mL, from 10 mg/mL to 20 mg/mL, from 10 mg/mL to 15 mg/mL, or from 15 mg/mL to 20 mg/mL.

59. (canceled)

60. (canceled)

61. (canceled)

62. (canceled)

63. (canceled)

64. (canceled)

65. (canceled)

66. The perfusion solution of claim 55, wherein the combination of salts comprises one or more of sodium ion, potassium ion, calcium ion, magnesium ion, hydrogen carbonate ion, chloride ion, sodium chloride, potassium chloride, calcium chloride, magnesium chloride, sodium dihydrogen phosphate, sodium bicarbonate, or sodium hydroxide.

67. The perfusion solution of claim 55, wherein the concentration of each salt corresponds to its normal serum concentration in human or animal blood.

68. (canceled)

69. (canceled)

70. (canceled)
